(19) United States
(12) Patent Application Publication
(10) Pub. No.: US 2007/0248672 A1
(43) Pub. Date: Oct. 25, 2007

(54) Indole and Azaindole Derivatives with Antitumor Action

(76) Inventors: Carlo Farina, Milano (IT); Stefania Gagliardi, Vimercate (IT); Paola Misiano, Goronzola (IT); Paolo Celestini, Meda (IT); Franco Zunino, Milano (IT)

Correspondence Address: Jay S Cinamon;Abelman Frayne and Schwab, 666 Third Avenue, New York, NY, 10017-5621, US

(21) Appl. No.: 11/579,237

(22) PCT Filed: Apr. 27, 2005
(86) PCT No.: PCT/EP05/51908

§ 371 Date: Oct. 30, 2006

(30) Foreign Application Priority Data
Apr. 30, 2004 (IT) MI2004A000874

(51) Int. Cl. A61K 31/404; A61K 31/40; A61K 31/4162; A61K 31/4196; A61K 31/421; A61K 31/495; A61K 31/5375; A61K 9/14; A61K 9/28; A61K 9/50; A61P 35/00; C07D 241/00; C07D 249/08; C07D 257/00; C07D 263/00; C07D 403/00; C07D 413/02; C07D 487/02

(52) U.S. Cl. 424/474; 424/489; 424/490; 514/235.5; 514/250; 514/254.01; 514/255.05; 514/364; 514/374; 514/381; 514/383; 514/396; 514/408; 514/415; 514/419; 544/141; 544/344; 544/373; 548/215; 548/250; 548/262.2; 548/312.1; 548/400; 548/469

(57) ABSTRACT

Indole and azaindole compounds useful for the treatment of solid tumours and tumours of the blood are described, they being particularly effective in the treatment of drug resistant tumours; these compounds are also able to synergistically enhance the activity of known antitumour drugs. They can hence be used either alone as antitumour agents or in association with known antitumour drugs. Processes for preparing the aforesaid compounds, which are partly new, and pharmaceutical compositions useful for the aforesaid treatments are also described.

### FIELD OF THE INVENTION

The present invention relates to the field of antitumour pharmacology. The preparation and the use of indole and azaindole derivatives in tumour treatment are described.

### PRIOR ART

Systemic tumour therapy involves the use of numerous drugs belonging to different classes. Despite substantial advances in tumour cell biology knowledge and the identification of possible cell targets useful for specific therapeutic interventions, the most effective drugs in current clinical use continue to be cytotoxic agents. These drugs react by interfering with critical cell processes such as DNA functions and cell replication, possessing high cytotoxic or antiproliferative potential. For this reason an important drawback of these drugs is their toxicity and low therapeutic index. However, the most critical limitation of conventional antitumour drugs is the phenomenon of cellular drug resistance which manifests itself in the majority of solid tumours. Indeed, with some exceptions (lymphomas, leukaemias, testicular tumours) in which conventional therapy results in a significant number of recoveries, human tumours in the advanced stage develop a state of resistance which is responsible for therapeutic failure. In these cases, even high dose intensive treatments and support therapies to reduce toxic effects have not produced advantageous therapeutic results. Therefore inherent resistance and acquired resistance, which manifests itself following an initial therapeutic benefit, are the principle problems of antitumour chemotherapy. In addition to the research of innovative molecules able to control tumour development using cytotoxic mechanisms or in a specific manner, a promising strategy for improving the effectiveness of pharmacological therapy appears to be the identification of molecules that can block the defense and survival abilities of the tumour during cytotoxic therapy treatment. It must therefore be presumed that a rational combination of cytotoxic drugs with molecules capable of interfering with relevant processes and/or defense mechanisms can substantially increase the chemosensitivity of tumour cells.

Drug resistance of tumour cells is a complex and multifactoral phenomenon. Some specific changes in the tumour cell can modify the expression of a drug target (for example, DNA topoisomerase) or can increase the capacity for repairing cytotoxic damage or can reduce susceptibility to apoptosis (for example via the overexpression of antiapoptotic factors). All these changes are directed to increase the survival ability of tumour cells. In addition the tumour cell, during the progression process, increases its defense abilities allowing it to survive and proliferate in unfavourable stressful conditions, such as the hypoxic/acid environment typical of the bulky masses of solid tumours, and to tolerate potentially lethal damage such as genotoxic damage. The expression of various defense factors (transport system, vacuolar ATPase) which play a role in reducing intracellular concentration of the drug or in its sub-cellular compartmentalization to hinder the interaction of the drug with the intracellular target, characterise a phenotype with multiple resistance which is typical of intrinsic resistance. Therefore, a pharmacological intervention aimed at these defense mechanisms, using well tolerated agents, should produce significant therapeutic advantages, improving the effectiveness of the cytotoxic drug without a substantial increase in toxicity (Oxford Textbook of Oncology, Second Edition, 2002, editors R. L. Souhami, L Tannock, P. Hohenberger, J. C. Horiot, Oxford University Press).

The phenomenon of drug resistance (multi-drug resistance-MDR) in tumour cells is therefore characterised by the development of a resistance to drug treatment, and is the major obstacle to chemotherapy.

A large amount of clinical evidence (Hirose M., J. Med. Invest 50, 126-135, 2003; Lin J. H., Drug Metab. Rev. 35, 417-454, 2003) shows that the MDR phenotype in tumours is associated with overexpression of proteins belonging to the ABC transporter family (P-glycoprotein or PgP, MDR, MRP, BCRP, etc.) which causes a reduction in the accumulation of a range of cytotoxic agents. The MDR phenomenon can be associated with expression changes of other proteins found on the cell membrane or within the tumour cell, such as DNA topoisomerase II (Jarvinen T. A., Breast Cancer Res. Treat 78, 299-311, 2003), glutathione S-transferase (Townsend D. M., Oncogene 22, 7369-7375, 2003), catalase (Tome M. E., Cancer Res., 61, 2766-2773, 2001) and vacuolar ATPase (V-ATPase) (Torigoe T., J. Biol. Chem. 277, 36534-36543, 2002).

Highly effective compounds have recently been found in the so called “unusual macrolides” class (bafilomycin A1 and concanamycin) and the macrolides derived from salicylic acid (salicylhalamide, lobatamide, oximidine and apicularen). Recent data in the literature demonstrate that these products can inhibit tumour cell growth in vitro (Boyd M. R., J. P. E. T. 297, 114-120, 2001; Bowman E. J., J. Biol. Chem. 278, 44147-44152, 2003). In-depth research on these products has proved to be extremely difficult due to the unavailability of sufficient amounts of the natural products and because of the fact that the synthesis processes to obtain them, if available, are lengthy, very complicated and very expensive. These difficulties, together with the known intrinsic toxicity of some of the aforementioned macrolides, limit their potential use in therapy.

The patent application EP 0449196 A2 describes some indole derivatives as bone resorption inhibitors useful for treating osteoporosis.

### SUMMARY

We have now discovered that the indole compounds of formula (I), 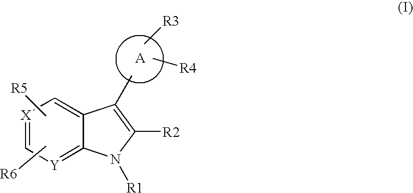
wherein:
R1 is chosen from H, alkyl, arylalkyl, hydroxyalkyl, alkoxyalkyl, aminoalkyl, alkylaminoalkyl, dialkylaminoalkyl, alkylCOOalkyl, alkoxycarbonylalkyl, aminocarbonylalkyl, alkylaminocarbonylalkyl, dialkylaminocarbonylalkyl, alkylCONalkyl, cyanoalkyl, or a group R′R″Nalkyl, in which R′ and R″, together with the nitrogen atom to which they are attached, may form a 5, 6 or 7 membered ring, optionally containing a heteroatom chosen from O, S and N, and where said N atom may be substituted by alkyl;
R2 is chosen from alkyl, alkenyl, aryl, heterocyclyl optionally substituted by alkyl or aryl, acid, ester, amide, nitrile, aminoalkyl, alkylaminoalkyl, dialkylaminoalkyl, hydroxyalkyl, CH₂NHCOCH₃, CONHSO₂CH₃, alkoxycarbonylalkyl, alkoxycarbonylalkenyl; or R1 and R2 together form a 5, 6 or 7 membered ring containing optionally a heteroatom chosen from O, S, N and containing optionally a carbonyl function which can be attached to any carbon atom of said ring, and where said N atom may be substituted by alkyl, aryl, arylalkyl, heteroaryl, alkylsulfonyl, arylsulfonyl, alkylcarbonyl, arylcarbonyl, alkylaminocarbonyl, arylaminocarbonyl;
R3, R4, R5, R6 each independently represent H, alkyl, alkoxy, hydroxy, halogen, trifluoromethyl, trifluoromethyloxy;
X and Y each independently represent carbon or nitrogen;
A is chosen from a phenyl or a heterocyclic ring with 5 or 6 members containing up to two heteroatoms chosen from nitrogen, oxygen and sulfur,
are inhibitors of vacuolar ATPase, possessing marked antiproliferative activity. They can therefore be used in the treatment of solid tumours and tumours of the blood such as leukaemias. This use is particularly indicated in tumours associated with the resistance phenomenon. In addition, the aforesaid compounds are able to enhance, in doses lower than those proven to be pharmacologically active, the activity of known cytotoxic agents. They can therefore either be used alone as antitumour drugs or in synergy with the action of known tumour agents. The compounds of formula (I) are partly new and partly described in the aforementioned patent application EP 0449196.

Additionally, the present application describes processes for preparing said compounds, their use in the treatment of tumours and resistance to antitumour drugs and their use as enhancers of the action of antitumour drugs. Moreover, pharmaceutical compositions containing the compounds of formula (I) are described, possibly associated with known antitumour drugs the action of which is to be enhanced.

The compounds of the present invention are also useful as radiosensitizers to reduce resistance to radiation therapy, a well known phenomenon occurring in many tumors.

Furthermore, the ability of compounds of the present invention to reduce in vitro chemoinvasion and in vivo metastasis, alone and in combination with known antitumor drugs, represents an excellent characteristic for a new antitumor agent, considering that metastasis is one of the major causes of death from cancer.

### DESCRIPTION OF THE FIGURES

FIG. 1: results of the co-treatment (72 hours) of HT29 cells with topotecan and compound of example 1 (representation according to Kern with illustration of synergism);

▪: Topotecan+compound of example 1 at 4 μM (124%);

◯: Topotecan+compound of example 1 at 8 μM (93%);

●: Topotecan+compound of example 1 at 4 μM (101%);

Δ: Topotecan+compound of example 1 at 8 μM (129%);

▴: Topotecan+compound of example 1 at 4 μM (135%)

FIG. 2: results of the co-treatment (72 hours) of HT29/Mit resistant cells with topotecan and compound of example 1 (representation according to Kern with illustration of synergism);

◯: Topotecan+compound of example 10.at 1 μM (78%);

●: Topotecan+compound of example 1 at 0.01 μM (122%).

FIG. 3: activity of the combination of topotecan and compound of example 1 in HT29/Mit xenograft mice model;

X: controls

◯: Topotecan (1 mg/kg) p.o.

Δ: Topotecan (2 mg/kg) p.o.

●: Topotecan (1 mg/kg) p.o.+compound of the example 1 (30 mg/kg) p.o.

▾: Topotecan (2 mg/kg) p.o.+compound of the example 1 (30 mg/kg) p.o.

### DETAILED DESCRIPTION OF THE INVENTION

In all the alkyl substituents of formula (I), and in those containing an alkyl group (for example hydroxyalkyl, alkylaminoalkyl), the alkyl residue can be indifferently linear, branched or cyclic, preferably a C1-C8 alkyl, more preferably C1-C4.

In all the alkenyl substitutents or those containing an alkenyl group (for example alkoxycarbonylalkenyl), the alkenyl residue can be indifferently linear, branched or cyclic, preferably a C1-C8 alkyl, more preferably C1-C4.

In the case of cyclic alkyls or alkenyls it is of course intended that the minimum number of carbon atoms cannot be less than 3.

In all the aryl substituents of formula (I), and in those containing an aryl group (for example arylalkyl), the aryl residue is preferably a phenyl.

The term “acid groups” means COOH groups. The term “ester groups” means COOR groups where R is an alkyl as aforedefined. The term “alkoxy groups” means OR groups where R is an alkyl as aforedefined. The term “amide groups” means CONR′R″ groups where R′ and R″ are H or an alkyl as aforedefined, or R′ and R″, together with the nitrogen atom to which they are attached may form a 5, 6 or 7 membered ring, optionally containing a heteroatom chosen from O, S and N.

More specifically, when R1 is an alkyl, it is preferably Me, Et or Pr; when R1 is an arylalkyl, it is preferably benzyl; when R1 is a hydroxyalkyl, it is preferably hydroxyethyl or hydroxypropyl; when R1 is alkoxyalkyl, it is preferably methoxyethyl; when R1 is an aminoalkyl, it is preferably aminoethyl; when R1 is an alkylaminoalkyl, it is preferably methylaminoethyl; when R1 is a dialkylaminoalkyl, it is preferably dimethylaminoethyl or dimethylaminopropyl; when R1 is alkoxycarbonylalkyl, it is preferably CH₂COOMe₂; when R1 is aminocarbonylalkyl, it is preferably CH₂CONH₂; when R1 is alkylaminocarbonylalkyl, it is preferably CH₂CONHMe; when R1 is dialkylaminocarbonylalkyl, it is preferably CH₂CONMe₂; when R1 is a cyanoalkyl, it is preferably CH₂CN; when R1 is R′R″Nalkyl, it is preferably pyrrolidinylethyl, morpholinylethyl or N-methylpiperazinylethyl.

When R2 is an alkoxycarbonylalkyl, it is preferably CH₂—CH₂COOEt; when R2 is an ester, it is preferably COOMe or COOEt, when R2 is an alkylaminoalkyl, it is preferably methylaminomethyl; when R2 is a heterocyclyl, it is preferably a 5-membered heterocycle containing from 2 to 4 heteroatoms chosen from N and O, more preferably a diazole, triazole, tetrazole or oxadiazole, which may be substituted with an alkyl or aryl group.

When R1 and R2 together form a 6 membered ring, the resulting compound is preferably a substituted 1,2,3,4-tetrahydro-pyrazino[1,2-a]indole, 3,4-dihydro-2H-pyrazino[1,2-a]indol-1-one, 1,2-dihydro-pyrazino[1,2-a]indol-3-one or 3,4-dihydro-1H-[1,4]oxazino[4,3-a]indole

When one or more of R3, R4, R5, R6 represents an alkyl, the alkyl group is preferably Me, Et; when they represent an alkoxy, the alkoxy group is preferably OMe, OEt; when they represent a halogen, it is preferably Cl or F. An aspect of the invention consists of the use of the compounds of formula (I) as aforedescribed in the preparation of a drug useful as an antitumour agent. The invention comprises in addition a method for the treatment of tumours, characterised by the administration of a compound of formula (I) to a patient requiring it.

The use/method of antitumour treatment is particularly indicated, although not exclusively, for the treatment of those tumours that have already developed resistance to conventional antitumour therapy or which are particularly open to the development of resistance; it particularly concerns tumours with a high level of expression in the transport systems responsible for the MDR phenomenon (multi-drug resistance or cross resistance), such as BCRP and PgP; examples of those tumours particularly exposed to the development of resistance are tumours of the digestive system such as carcinomas of the stomach, colon, liver and pancreas, tumours of the urinary system, tumours of the central nervous system such as neuroblastoma and glioma, tumour of the breast, of the bones and melanoma (Ouar Z., Biochem. J. 370, 185-193, 2003; Ohta T., J. Pathol. 185, 324-330, 1998, Nakashima S., J. Biochem.-Tokyo-134, 359-64, 2003; Altan N., J. Exp. Med. 187, 1583-1598, 1998; Martinez-Zaguilan R., Biochem. Pharmacol. 57, 1037-1046, 1999).

Moreover, the compounds of the present invention are able to enhance the activity of known cytotoxic agents at doses lower than those demonstrated to be pharmacologically active.

Examples of conventional antitumour drugs which can give rise to various manifestations of drug resistance and which can benefit combined treatment with the compounds of formula (I) are anthracyclines (for example doxorubicin, epirubicin, mitoxantrone), camptothecins (for example topotecan, irinotecan), platinum compounds (for example cisplatin, carboplatin) and taxans (for example taxol and taxotere).

The antitumour use claimed, therefore also encompasses using said compounds to synergistically enhance the action of known antitumour agents and to treat the phenomenon of drug resistance having developed following antitumour therapies. A further aspect of the invention consists of pharmaceutical compositions comprising one or more compounds of formula (I) as aforedefined possibly combined with one or more antitumour agents, and in the presence of suitable pharmaceutical excipients. The dosage units of these pharmaceutical compositions contain the compound of formula (I) in a quantity between 1 and 1000 mg; said units are administered so that in the patient dosages per Kg are achieved which are preferably within the aforementioned range. The antitumour agent present in the compositions with the compound of formula (I), is used in the normal amounts at which it is already known to be active, or in a possibly lower amount by virtue of the synergistic enhancement effect acheived by the present invention. Non-limiting reference limits for antitumour drug content, combined with the compound of formula (I) in the dosage unit, are between 0.1 and 1000 mg.

The pharmaceutical compositions of the invention can be adapted for the various administration routes, and can be provided for example in the form of injectable solutions, solutions for infusion, solutions for inhalation, suspensions, emulsions, syrups, elixirs, drops, suppositories, possibly coated pills, hard or soft capsules, microcapsules, granules, dispersible powders etc.

The excipients contained in these compositions are those commonly used in pharmaceutical technology, and can be used in the manner and quantity commonly known to the expert of the art.

Solid administration forms, such as pills and capsules for oral administration, are normally supplied in dosage units. They contain conventional excipients such as binders, fillers, diluents, tabletting agents, lubricants, detergents, disintegrants, colorants and wetting agents and can be coated in accordance with methods well known in the art.

The fillers include for example cellulose, mannitol, lactose and similar agents. The disintegrants include starch, polyvinylpyrrolidone and starch derivatives such as sodium starch glycolate; the lubricants include, for example, magnesium stearate; the wetting agents include for example sodium lauryl sulfate. These solid oral compositions can be prepared with conventional mixing, filling or tabletting methods. The mixing operations can be repeated to disperse the active agent in compositions containing large quantities of fillers. These are conventional operations.

The liquid preparations can be provided as such or in the form of a dry product to be reconstituted with water or with a suitable carrier at the time of use. These liquid preparations can contain conventional additives such as suspending agents, for example sorbitol, syrup, methylcellulose, gelatin, hydroxyethylcellulose, carboxymethylcellulose, aluminium stearate gel or hydrogenated edible fats, emulsifying agents, for example lecithin, sorbitan monooleate, or acacia; non aqueous carriers (which can include edible oil) for example almond oil, fractionated coconut oil, oily esters such a glycerin esters, propylene glycol or ethyl alcohol; preservatives, for example methyl or propyl p-hydroxybenzoate or sorbic acid and if desired, conventional flavours or colorants. The oral formulations also include sustained release conventional formulations, such as enteric coated pills or granules.

For parenteral administration, fluid dosage units can be prepared containing the compound and a sterile carrier. The compound, depending on the carrier and concentration, can be suspended or dissolved. The parenteral solutions are normally prepared by dissolving the compound in a carrier and sterilizing by filtration, before filling suitable vials or ampoules and sealing. Adjuvants such as local anesthetics, preservatives and buffering agents can be advantageously dissolved in the carrier. In order to increase stability, the composition can be frozen after filling the vial and the water removed under vacuum. The parenteral suspensions are prepared essentially in the same way, with the difference that the compound can be suspended rather than dissolved in the carrier, and can be sterilized by exposure to ethylene oxide prior to being suspended in the sterile carrier. A surfactant or humectant can be advantageously included in the composition to facilitate uniform distribution of the compound of the invention.

As is the common practise, the compositions are normally accompanied by written or printed instructions, for use in the treatment concerned.

A part of the compounds of formula (I) are new. These concern the compounds of formula (I) in which X, Y, A, R1, R2, R3, R4, R5, R6 have the previously defined meanings, with the exception of compounds in which X, Y are carbon, A is phenyl, R1 is H or alkyl and, simultaneously, R2 is chosen from acid, ester, amide or hydroxyalkyl.

A sub-group of new compounds in accordance with the invention consists of compounds of formula (I) in which X, Y, A, R1, R2, R3, R4, R5, R6 have the previously defined meanings, with the exception of the compounds in which X, Y are carbon, A is phenyl, R1 is H or alkyl and, simultaneously, R2 is chosen from acid, ester, amide, hydroxyalkyl, CH₂NHCOCH₃, CONHSO₂CH₃, alkoxycarbonylalkyl or alkoxycarbonylalkenyl.

The invention also includes the use of the new compounds of the invention for therapy, particularly for the aforementioned antitumour treatments, as well as pharmaceutical compositions which comprise them, possibly in association with known antitumour drugs.

The compounds of formula (I) can be obtained by alkylation of the compound of formula (II) 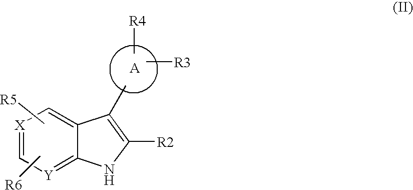
wherein X, Y, A, R₂, R₃, R₄, R₅ and R₆ are as aforedefined for the compounds of formula (I), with a halide of formula R₁X₁ wherein R₁ is as defined for formula (I) and X₁ is bromine, chlorine or iodine.

The reaction is conducted under conventional alkylation conditions, for example in an aprotic solvent such as tetrahydrofuran or dimethylformamide in the presence of a suitable base such as sodium or potassium hydride or the lithium salt of a secondary amine. The reaction temperature can be between 25° C. and 50° C.; the reaction time is comprised for example between 30 minutes and 24 hours.

The compounds R₁X₁ are known compounds, available commercially or preparable by methods used to produce known compounds.

The compound of formula (III), 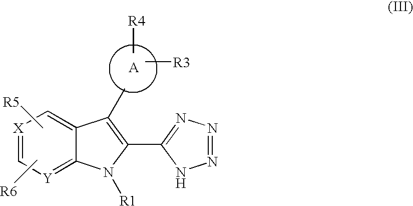
wherein X, Y, A, R1, R3, R4, R5 and R6 are as defined for the compounds of formula (I), can be obtained from the compound of formula (IV) 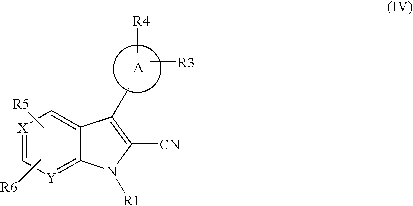
wherein X, Y, A, R1, R3, R4, R5 and R6 are as defined for the compound of formula (I). The heterocycle formation reaction can be conducted by reacting with Bu₃SnN₃ at 120° C. in a solvent such as DMF or without solvent followed by hydrolysis of the corresponding stannane with aqueous HCl.

Compounds of formula (IV) may be obtained by dehydration of compounds of formula (V) 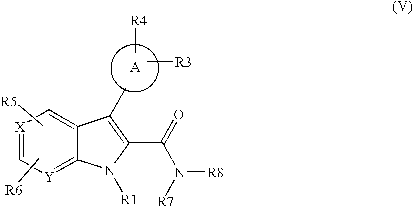
wherein X, Y, A, R1, R3, R4, R5 and R6 are as defined for formula (I), R7 and R8 are hydrogen, alkyl or, together with the nitrogen atom to which they are attached, may form a 5, 6 or 7 membered ring optionally containing a heteroatom chosen from N, O or S, with the proviso that the described process is possible when both R7 and R8 are hydrogen.

Dehydration can be conducted with all the reagents commonly used for dehydration of primary amides, for example trifluoroacetic anhydride, at a suitable temperature for forming the desired product, for example from 0° C. to ambient temperature, in the presence of suitable solvents such as pyridine or dioxane.

The compound of formula (V) can be obtained from the compound of formula (VI), 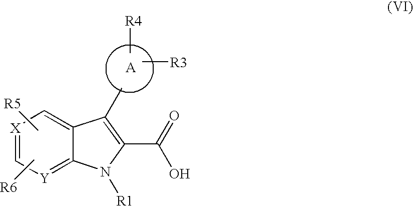
wherein X, Y, A, R1, R3, R4, R5 and R6 are as defined for the compounds of formula (I), and an amine of formula HNR7R8 following the reaction process described in EP 449196. Compounds of formula HNR7R8 are known compounds which are commercially available or can be prepared by using methods analogous to those used for preparing known compounds; for example the methods described in Chemistry of the Amino Group, Patais (Ed.), Interscience, New York 1968; Advanced Organic Chemistry, March J, John Wiley & Sons, New York, 1992.

The compound of formula (VI) can be obtained from the compound of formula (VII), 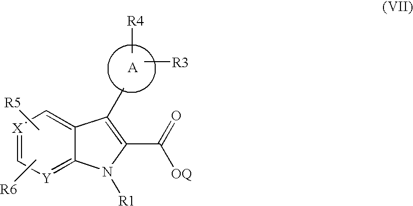
wherein X, Y, A, R1, R3, R4, R5 and R6 are as defined for the compounds of formula (I), and Q is C₁₋₄alkyl, following the reaction process described in EP 449196. The compound of formula (VII) can be obtained from the compound of formula (VIII), 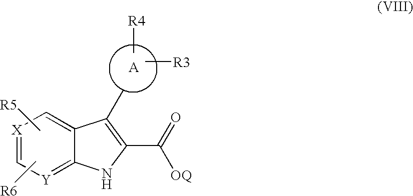
wherein X, Y, A, R3, R4, R5 and R6 are as defined for the compounds of formula (I), and Q is as defined for compounds of formula (VII), following the process described for preparation of the compounds of formula (I).

Compounds of formula (VIII) may be obtained following processes described in EP 449196.

Alternatively, compounds of formula (VIII) may be obtained reacting compounds of formula (IX), 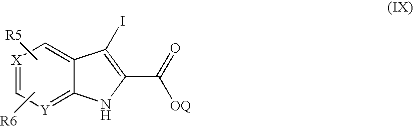
wherein X, Y, R5 and R6 are as defined for formula (I) and Q is C₁₋₄ alkyl, with boronic acids of formula (X), 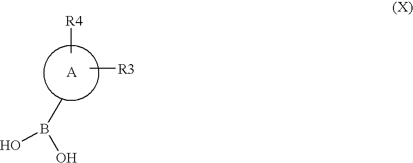
wherein A, R3 and R4 are as defined for formula (I), and a palladium catalyst such as Pd(PPh₃)₄ or Pd(dppf)Cl₂ under Suzuki coupling conditions, as described for example in Chem. Rev. 1995, 95, 2457-2483

Compounds of formula (IX) are either known or commercially available, or may be prepared as described for example in Chem. Pharm. Bull. 1988, 36, 2248-2252. Compounds of formula (X) are either known or commercially available, or may be prepared as described for example in Chem. Rev. 1995, 95, 2457-2483.

The compound of formula (XI), 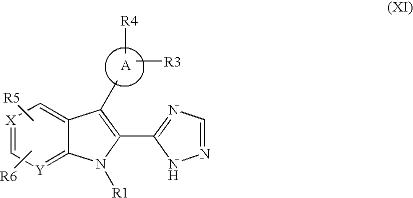
wherein X, Y, A, R1, R3, R4, R5 and R6 are as defined for compounds of formula (I), can be obtained from the compound of formula (V) in which R7 and R8 are hydrogen, as described in J. Org. Chem. 1979, 44, 4160-4164 for the generic synthesis of 1,2,4 triazoles.

The compound of formula (XII), 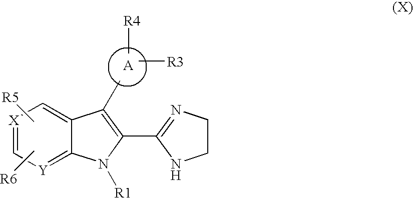
wherein X, Y, A, R1, R3, R4, R5 and R6 are as defined for the compounds of formula (I), can be obtained from the compound of formula (VI) as described in J. Med. Chem. 1987, 30, 1555-1562.

The compound of formula (XIII), 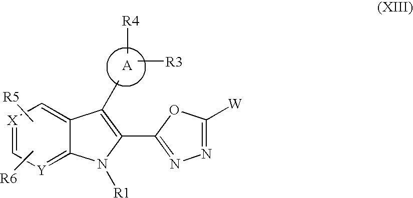
wherein X, Y, A, R1, R3, R4, R5 and R6 are as defined for the compounds of formula (I), and W is Hydrogen or an Alkyl Group as Previously Defined or an Aryl group, can be obtained from the compound of formula (XIV). 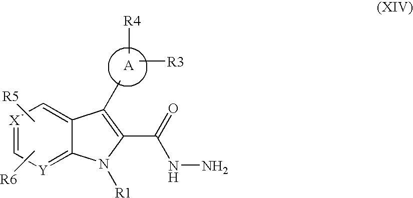
wherein X, Y, A, R1, R3, R4, R5 and R6 are as defined for the compounds of formula (I), as described in Bull. Soc. Chim. Fr. 1977, 333-336.

The compound of formula (XIV) can be obtained reacting the compound of formula (VI) with hydrazine following the general amidation conditions described in EP449196.

The compound of formula (XV), 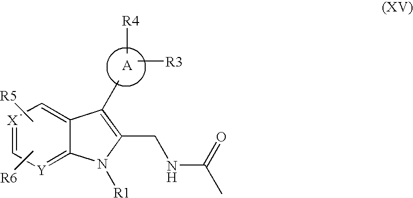
wherein X, Y, A, R1, R3, R4, R5 and R6 are as defined for the compounds of formula (I) can be obtained from the compound of formula (XVI) 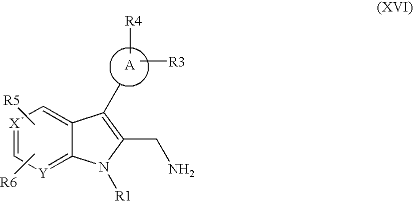
wherein X, Y, A, R1, R3, R4, R5 and R6 are as defined for the compounds of formula (I). The reaction can be conducted by reacting the compound of formula (XVI) with acetylating agents such as acetyl chloride or acetic anhydride, in the presence of an organic or inorganic base, for example triethylamine (TEA) in a chlorinated or aprotic solvent, for example methylene chloride, at any temperature which supplies a suitable percentage of the required product, for example at ambient temperature.

The compound of formula (XVI) can be obtained from the compound of formula (V) in which R7 and R8 are hydrogen, by reduction with a suitable reducing agent, for example with an aluminium hydride or a borane in an ether solvent, for example tetrahydrofuran, at any temperature which provides a suitable percentage of the required product, for example from ambient temperature to 50° C.

The compound of formula (XVII), 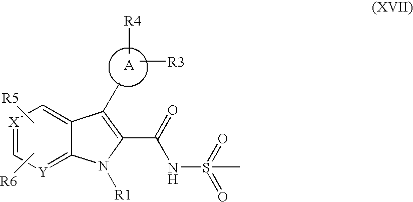
wherein X, Y, A, R1, R3, R4, R5 and R6 are as defined for the compounds of formula (I), can be obtained from the compound of formula (VI) following the process described in Bioorg. Med. Chem. Lett. 2002, 12, 125-128. 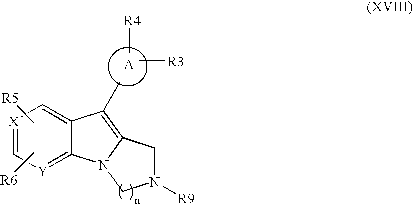
wherein X, Y, A, R3, R4, R5 and R6 are as defined for the compounds of formula (I), R9 is H or is an alkyl group as previously defined and n is 1, 2, or 3, may be obtained by reduction of compounds of formula (XIX) 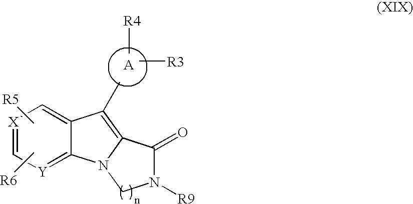
wherein X, Y, A, R3, R4, R5 and R6 are as defined for the compounds of formula (I), R9 is H or is an alkyl group as previously defined and n is 1, 2, or 3, using a suitable reducing agent, for example an aluminium hydride or a borane in an ether solvent, for example tetrahydrofuran, at any temperature which provides a suitable percentage of the required product, for example from ambient temperature to 50° C.

Compounds of formula (XIX) in which R9 is alkyl may be obtained alkylating compounds of formula (XIX) in which R9 is hydrogen with alkyl halides of formula R9-X₁, in which R9 is alkyl and X₁ is Cl, Br, I, under conditions described above for the preparation of compounds of formula (I).

Compounds of formula (XIX) in which R9 is hydrogen or alkyl may be obtained from compounds of formula (XX) 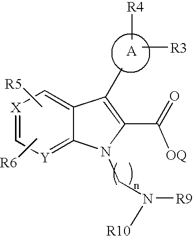
wherein X, Y, A, R3, R4, R5 and R6 are as defined for the compounds of formula (I), Q is as previously defined, n is 1, 2, or 3, R9 is as defined for the compounds of formula (XIX) and R10 has the same meanings of R9, or R9 and R10 taken together with the nitrogen atom to which they are attached may form a 5, 6 or 7 membered heterocyclic ring optionally containing a heteroatom chosen from N, O and S, and when said heteroatom is N, it may be substituted by alkyl, with the proviso that the described process is possible when R10 is hydrogen.

The described process is typically a transamidation reaction using a suitable reagent for direct amidation, for example trimethyl aluminium in toluene, at any temperature which provides a suitable percentage of the required product, for example from 80° C. to 120° C.

Compounds of formula (XX) may be obtained from compounds of formula (XXI) 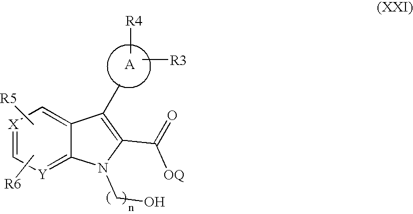
wherein X, Y, A, R3, R4, R5 and R6 are as defined for the compounds of formula (I), Q is as previously defined and n is 1, 2, or 3, by transforming the hydroxy group into a suitable leaving group such as bromide, iodide, mesylate or tosylate, followed by reaction with an amine of formula R9R10NH, wherein R9 and R10 are as defined for the compounds of formula (XX)

Compounds of formula (XXI) may be obtained from compounds of formula (VII) under conditions described above for compounds of formula (I).

Compounds of formula (XX) in which both R9 and R10 are hydrogen may be obtained from compounds of formula (XXII) 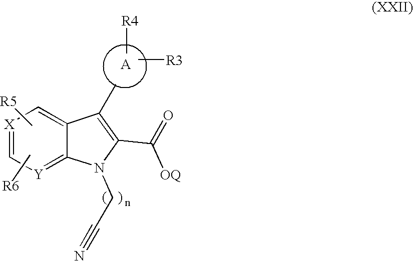
wherein X, Y, A, R3, R4, R5 and R6 are as defined for the compounds of formula (I), Q is as defined previously and n is 1, 2, or 3, by catalytic hydrogenation in an acid environment. Catalytic hydrogenation can be conducted in an alcoholic solvent or ethyl acetate and using a suitable catalyst, for example palladium supported on carbon under a suitable hydrogenation pressure, for example 45 psi.

Compounds of formula (XXII) can be obtained by alkylation of the compound of formula (VII), wherein X, Y, A, R3, R4, R5 and R6 are as defined for the compounds of formula (I), with haloalkylnitriles, following the general methodology described for the preparation of the compounds of formula (I).

Compounds of formula (XXIII) 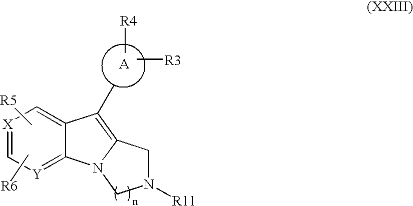
wherein X, Y, A, R3, R4, R5 and R6 are as defined for the compounds of formula (I), n is 1, 2, or 3, R11 is —SO₂R12, —COR12 or —CONHR12 and R12 is alkyl as previously defined or optionally substituted aryl, may be obtained reacting compounds of formula (XVIII) in which R9 is hydrogen, with compounds of formula R12SO₂Cl, R12COCl, R12COOCOR12 or R12NCO, in a suitable solvent such as dichloromethane and in presence, if necessary, of a suitable base such as triethylamine or pyridine.

Compounds of formula R12SO₂Cl, R12COCl, R12COOCOR12 or R12NCO are known or commercially available or may be prepared as described in standard reference texts of organic synthesis such as J. March, Advanced Organic Chemistry, 3rd Edition (1985), Wiley Interscience.

The compound of formula (XXIV), 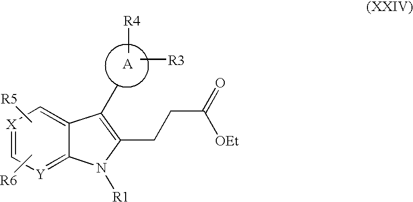
wherein X, Y, A, R1, R3, R4, R5 and R6 are as defined for the compounds of formula (I) can be obtained from the compound of formula (XXV), 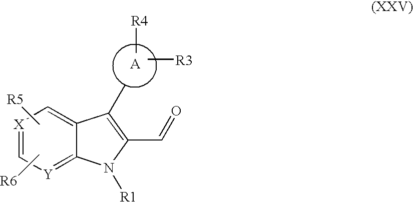
wherein X, Y, A, R1, R3, R4, R5 and R6 are as defined for the compounds of formula (I,) by Horner-Emmons reaction followed by catalytic hydrogenation of the double bond. The Horner-Emmons reaction can be conducted with an ethyl alkylphosphonoacetate, a base such as a lithium, sodium or potassium hydride, a sodium or potassium alcoholate, lithium alkyl derivatives and the like, in an ether solvent.

Catalytic hydrogenation can be conducted in an alcoholic solvent or ethyl acetate and using a suitable catalyst, for example palladium supported on carbon under a suitable hydrogen pressure, for example 45 psi.

The compound of formula (XXV) can be obtained by the oxidation of the compound of formula (XXVI), 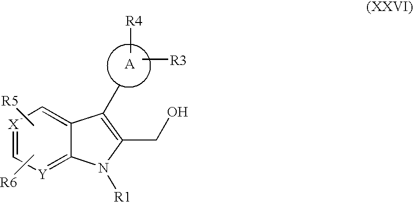
wherein X, Y, A, R1, R3, R4, R5 and R6 are as defined for the compounds of formula (I), using one of the known reactions in the literature for oxidizing primary and benzyl alcohols. The oxidation can be conducted with conventional oxidizing agents such as manganese dioxide, pyridinium-chloro-chromate, dimethylsulfoxide and oxalyl chloride. The solvent can be a chlorinated solvent or an ether solvent.

The compound of formula (XXVI) can be obtained by reduction of the compound of formula (VII) using a suitable reducing agent, for example an aluminium hydride or a borane in an ether solvent, for example tetrahydrofuran, at any temperature which provides a suitable percentage of the required product, for example from ambient temperature to 50° C.

The compound of formula (XXVII), 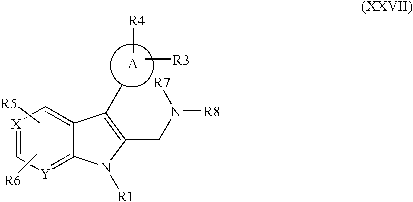
wherein X, Y, A, R1, R3, R4, R5 and R6 are as defined for the compounds of formula (I) and R7 and R8 are as previously defined in the description of formula (V), can be obtained from the compound of formula (XXV) by reductive amination reaction with the compounds of formula HNR7R8 and a boron hydride such as sodium cyanoborohydride and sodium triacetoxy borohydride and an alcoholic solvent such as methanol at a temperature which supplies a suitable percentage of the required product, for example ambient temperature.

Compounds of formula (XXVIII) 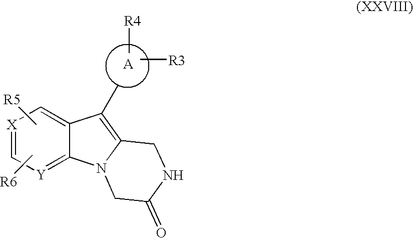
wherein X, Y, A, R3, R4, R5 and R6 are as defined for the compounds of formula (I), may be obtained by reaction of compounds of formula (XXIX) 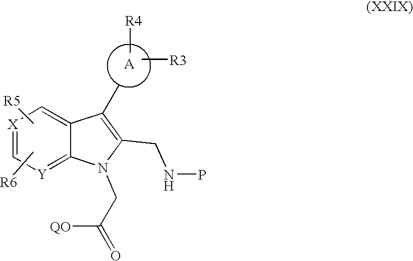
wherein X, Y, A, R3, R4, R5 and R6 are as defined for the compounds of formula (I), P is a suitable protecting group such as trityl and Q is as previously defined, by simultaneous deprotection and cyclization after treatment with a suitable deprotecting agent, such as trifluoroacetic acid.

Compounds of formula (XXIX) may be obtained from compounds of formula (XXX) 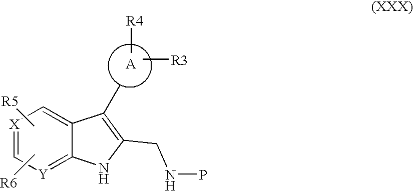
wherein X, Y, A, R3, R4, R5 and R6 are as defined for the compounds of formula (I), and P is as previously defined, by alkylation with BrCH₂COOQ under conditions described for the preparation of compounds of formula (I)

Compounds of formula (XXX) may be obtained from compounds of formula (XXVII) in which R1, R7 and R8 are hydrogen, by protection with a suitable agent, such as trityl chloride.

Compounds of formula (XXXI) 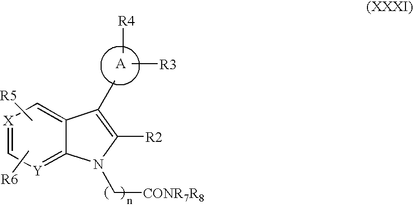
wherein X, Y, A, R2, R3, R4, R₅ and R₆ are as defined for the compounds of formula (I), R7 and R₈ are as previously defined and n is 1, 2, or 3, may be prepared from compounds of formula (XXXII) 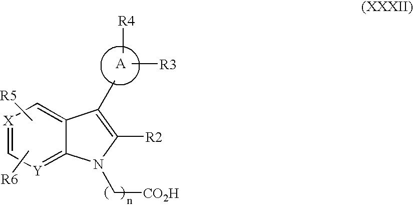
wherein X, Y, A, R2, R3, R4, R5 and R6 are as defined for the compounds of formula (I) and n is 1, 2, or 3, by activation of the carboxylic moiety with for example thionyl chloride or oxalyl chloride or carbonyldiimidazole and reaction with an amine of formula R7R8NH.

Compounds of formula (XXXII) may be prepared from compounds of formula (XXXIII) 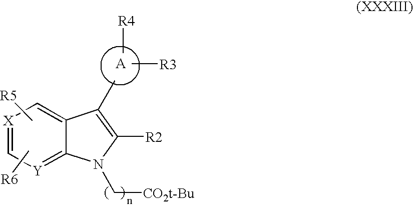
wherein X, Y, A, R2, R3, R4, R5 and R6 are as defined for the compounds of formula (I) and n is 1, 2, or 3 by deprotection with for example trifluoroacetic acid or HCl/diethyl ether.

Compounds of formula (XXXIII) may be prepared from compounds of formula (II) as described above for compounds of formula (I)

Alternatively, compounds of formula (XXXI) in which R7 and R8 are hydrogen may be prepared on solid phase, reacting a Sieber resin with bromoacetic acid and a suitable coupling agent such as dicyclohexylcarbodiimide, then adding a suitable base such as sodium hydride and a compound of formula (II) in a suitable solvent such as DMF, and finally cleaving the product from the resin with a suitable cleaving reagent, such as a mixture of trifluoroacetic acid and triisopropylsilane in methylene chloride.

Compounds of formula (XXXIV) 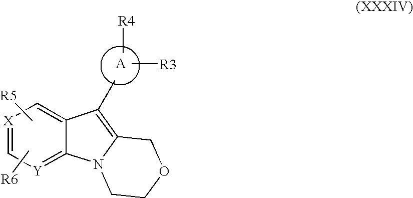
wherein X, Y, A, R3, R4, R5 and R6 are as defined for the compounds of formula (I), may be prepared from compounds of formula (XXXV) 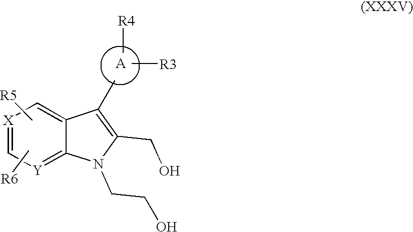
wherein X, Y, A, R3, R4, R5 and R6 are as defined for the compounds of formula (I), by simultaneous activation and ring closure with a suitable combination of an activating agent and a base, such as tosylimidazole and sodium hydride. Compounds of formula (XXXV) may be obtained from compounds of formula (XXXII), in which R2 is a group COOQ where Q is as defined previously, by reduction with a suitable reducing agent such as LiAlH₄ in a suitable solvent, such as THF.

Compounds of formula (XXXVI) 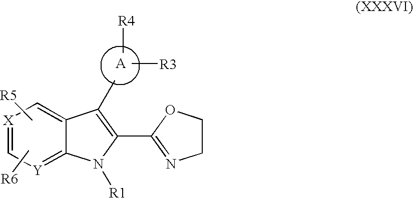
wherein X, Y, A, R1, R3, R4, R5 and R6 are as defined for the compounds of formula (I), may be prepared from compounds of formula (VI) by activation of the carboxylic moiety as described above for compounds of formula (XXXI) and formation of amide with simultaneous ring closure with 2-bromoethylamine.

Non-limiting examples of compounds of formula (I) useful for the purposes of the invention are the following:
5,6-Dimethoxy-3-(4-methoxy-phenyl)-1H-indole-2-carboxylic acid methylester
3-(3,4-Dimethoxy-phenyl)-5,6-dimethoxy-1H-indole-2-carboxylic acid methylester
5,6-Dimethoxy-3-phenyl-1H-indole-2-carboxylic acid methylester
5,6-Dimethoxy-3-phenyl-1H-indole-2-carboxylic acid ethylester
3-(4-Methoxy phenyl)-1H-indole-2-carboxylic acid methylester
1-Benzyl-5,6-dimethoxy-3-(4-methoxyphenyl)-1H-indole-2-carboxylic acid methylester
5,6-Dimethoxy-1-methoxycarbonylmethyl-3-(4-methoxyphenyl)-1H-indole-2-carboxylic acid methylester
1-Dimethylcarbamoylmethyl-5,6-dimethoxy-3-(4-methoxyphenyl)-1H-indole-2-carboxylic acid methylester
5,6-Dimethoxy-3-(4-methoxyphenyl)-1-propyl-1H-indole-2-carboxylic acid methylester
1-Cyanomethyl-5,6-dimethoxy-3-(4-methoxyphenyl)-1H-indole-2-carboxylic acid methylester
1-(2-Dimethylaminoethyl)-5,6-dimethoxy-3-(4-methoxyphenyl)-1H-indole-2 carboxylic acid methylester hydrochloride
1-(2-Hydroxyethyl)-5,6-dimethoxy-3-(4-methoxyphenyl)-1H-indole-2-carboxylic acid methylester
5,6-Dimethoxy-3-(4-methoxyphenyl)-1H-indole-2-carboxylic acid
5,6-Dimethoxy-3-(4-methoxyphenyl)-1H-indole-2-carboxyamide
2-Aminomethyl-5,6-dimethoxy-3-(4-methoxyphenyl)-1H-indole hydrochloride
N-[5,6-dimethoxy-3-(4-methoxyphenyl)-1H-indol-2-ylmethyl]-acetamide
5,6-Dimethoxy-3-(4-methoxyphenyl)-1H-indole-2-carbonitrile
1-(2-Dimethylaminoethyl)-5,6-dimethoxy-3-(4-methoxy-phenyl)-1H-indole-2-carbonitrile hydrochloride
N-[5,6-dimethoxy-3-(4-methoxyphenyl)-1H-indole-2-carbonyl]-methanesulfonamide
[5,6-Dimethoxy-3-(4-methoxyphenyl)-1H-indol-2-yl]-methanol
[5,6-Dimethoxy-3-(4-methoxyphenyl)-1H-indol-2-ylmethyl]-methylamine hydrochloride
3-[5,6-dimethoxy-3-(4-methoxyphenyl)-1H-indol-2-yl]-propionic acid ethyl ester
5,6-Dimethoxy-3-(4-methoxyphenyl)-2-(2H-[1,2,4]triazol-3-yl)-1H-indole
2-(4,5-Dihydro-1H-imidazol-2-yl)-5,6-dimethoxy-3-(4-methoxy-phenyl)-1H-indole trifluoroacetate
5,6-Dimethoxy-3-(4-methoxyphenyl)-2-(1H-tetrazol-5-yl)-1H-indole
5,6-Dimethoxy-3-(4-methoxyphenyl)-2-[1,3,4]oxadiazol-2-yl-1H-indole
7,8-Dimethoxy-10-(4-methoxyphenyl)-3,4-dihydro-2H-pyrazino[1,2-a]indol-1-one
7,8-Dimethoxy-10-(4-methoxyphenyl)-1,2,3,4-tetrahydro-pyrazino[1,2-a]indole hydrochloride
5,6-Dimethoxy-3-phenyl-1H-indole-2-carboxylic acid
3-(4-Chlorophenyl)-5,6-dimethoxy-1H-indole-2-carboxylic acid methyl ester
3-(4-(Trifluoromethyl)phenyl)-5,6-dimethoxy-1H-indole-2-carboxylic acid methyl ester
5,6-Dimethoxy-3-p-tolyl-1H-indole-2-carboxylic acid methyl ester
3-(4-Fluorophenyl)-5,6-dimethoxy-1H-indole-2-carboxylic acid methyl ester
3-(2-Chlorophenyl)-5,6-dimethoxy-1H-indole-2-carboxylic acid methyl ester
3-(3-Chlorophenyl)-5,6-dimethoxy-1H-indole-2-carboxylic acid methyl ester
5-Chloro-3-phenyl-1H-indole-2-carboxylic acid ethyl ester
5-Fluoro-3-phenyl-1H-indole-2-carboxylic acid ethyl ester
5-Methoxy-3-phenyl-1H-indole-2-carboxylic acid ethyl ester
5,6-Dimethoxy-1-(2-methoxyethyl)-3-(4-methoxyphenyl)-1H-indole-2-carboxylic acid methyl ester
1-(2-Hydroxyethyl)-5,6-dimethoxy-3-phenyl-1H-indole-2-carbonitrile
1-(3-Hydroxypropyl)-5,6-dimethoxy-3-(4-methoxyphenyl)-1H-indole-2-carboxylic acid methyl ester
1-(2-Hydroxyethyl)-5,6-dimethoxy-3-(4-methoxyphenyl)-1H-indole-2-carbonitrile
2-(5,6-Dimethoxy-3-(4-methoxyphenyl)-2-(5-methyl-1,3,4-oxadiazol-2-yl)-1H-indol-1-yl)ethanol
1-(3-Hydroxypropyl)-5,6-dimethoxy-3-(4-methoxyphenyl)-1H-indole-2-carbonitrile
5,6-Dimethoxy-3-phenyl-1H-indole-2-carboxylic acid amide
5,6-Dimethoxy-3-phenyl-1H-indole-2-carboxylic acid dimethylamide
(5,6-Dimethoxy-3-phenyl-1H-indol-2-yl)-morpholin-4-yl-methanone
5,6-Dimethoxy-3-phenyl-1H-indole-2-carboxylic acid methylamide
5,6-Dimethoxy-3-phenyl-1H-indole-2-carbonitrile
5,6-Dimethoxy-3-phenyl-1-propyl-1H-indole-2-carbonitrile
1-(2-(Dimethylamino)ethyl)-5,6-dimethoxy-3-phenyl-1H-indole-2-carbonitrile hydrochloride
5,6-Dimethoxy-3-(4-methoxyphenyl)-1-(2-(pyrrolidin-1-yl)ethyl)-1H-indole-2-carboxylic acid methyl ester hydrochloride
1-(3-(Dimethylamino)propyl)-5,6-dimethoxy-3-(4-methoxyphenyl)-1H-indole-2-carboxylic acid methyl ester hydrochloride
5,6-Dimethoxy-3-(4-methoxyphenyl)-1-(2-(pyrrolidin-1-yl)ethyl)-1H-indole-2-carbonitrile hydrochloride
1-(3-(Dimethylamino)propyl)-5,6-dimethoxy-3-(4-methoxyphenyl)-1H-indole-2-carbonitrile
5,6-Dimethoxy-3-(4-methoxyphenyl)-1-(2-morpholinoethyl)-1H-indole-2-carbonitrile hydrochloride
2-(5,6-Dimethoxy-3-(4-methoxyphenyl)-2-(5-methyl-1,3,4-oxadiazol-2-yl)-1H-indol-1-yl)-N,N-dimethylethanamine
5,6-Dimethoxy-3-phenyl-2-(4H-1,2,4-triazol-3-yl)-1H-indole
3,4-Dihydro-7,8-dimethoxy-10-phenylpyrazino[1,2-a]indol-1(2H)-one
1-(2-Amino-ethyl)-5,6-dimethoxy-3-(4-methoxy-phenyl)-1H-indole-2-carboxylic acid methyl ester hydrochloride
7,8-Dimethoxy-10-phenyl-1,2,3,4-tetrahydro-pyrazino[1,2-a]indole hydrochloride
5,6-Dimethoxy-3-(4-methoxyphenyl)-1-[2-(4-methylpiperazin-1-yl)-ethyl]
1H-indole-2-carboxylic acid methyl ester dihydrochloride
5,6-Dimethoxy-3-(4-methoxyphenyl)-1-(2-morpholinoethyl)-1H-indole-2-carboxylic acid methyl ester hydrochloride
5,6-Dimethoxy-3-(4-methoxyphenyl)-1-(2-(4-methylpiperazin-1-yl)ethyl)-1H-indole-2-carbonitrile dihydrochloride
7,8-Dimethoxy-10-(4-methoxyphenyl)-2-methyl-1,2,3,4-tetrahydro-pyrazino[1,2-a]indole hydrocloride
7,8-Dimethoxy-10-(4-methoxyphenyl)-1,2-dihydropyrazino[1,2-a]indol-3-one
2-Methanesulfonyl-7,8-dimethoxy-10-(4-methoxyphenyl)-1,2,3,4-tetrahydro-pyrazino[1,2-a]indole
7,8-Dimethoxy-10-(4-methoxyphenyl)-2-(propane-2-sulfonyl)-1,2,3,4-tetrahydropyrazino[1,2-a]indole
7,8-Dimethoxy-10-(4-methoxyphenyl)-2-(toluene-4-sulfonyl)-1,2,3,4-tetrahydropyrazino[1,2-a]indole
1-[7,8-Dimethoxy-10-(4-methoxyphenyl)-3,4-dihydro-1H-pyrazino[1,2-a]indol-2-yl]-ethanone
7,8-Dimethoxy-10-(4-methoxyphenyl)-3,4-dihydro-1H-pyrazino[1,2-a]indole-2-carboxylic acid methylamide
2-Isopropyl-7,8-dimethoxy-10-(4-methoxyphenyl)-1,2,3,4-tetrahydro-pyrazino[1,2-a]indole hydrochloride
1-Carbamoylmethyl-5,6-dimethoxy-3-(4-methoxyphenyl)-1H-indole-2-carboxylic acid methyl ester
2-(4,5-Dihydrooxazol-2-yl)-5,6-dimethoxy-3-phenyl-1H-indole
5,6-Dimethoxy-3-(4-methoxyphenyl)-1-methylcarbamoylmethyl-1H-indole-2-carboxylic acid methyl ester
3,4-Dihydro-7,8-dimethoxy-10-(4-methoxyphenyl)-1H-[1,4]oxazino[4,3-a]indole
5,6-Dimethoxy-3-(4-methoxyphenyl)-2-(5-methyl-1,3,4-oxadiazol-2-yl)-1H-indole
5-Hydroxy-3-phenyl-1H-indole-2-carboxylic acid ethyl ester
3-Pyridin-3-yl-1H-indole-2-carboxylic acid ethyl ester
3-Phenyl-1H-indole-2-carboxylic acid ethyl ester
5,6-Dimethoxy-3-pyridin-4-yl-1H-indole-2-carboxylic acid ethyl ester

The experimental section which follows further illustrates the invention without limiting it.

Experimental Part

### Chemical Synthesis

### EXAMPLE 1

### 5,6-Dimethoxy-3-(4-methoxy-phenyl)-1H-indole-2-carboxylic acid methyl ester

The compound was obtained as described in EP 449196

### EXAMPLE 2

### -3-(3,4-Dimethoxyphenyl)-5,6-dimethoxy-1H-indole-2-carboxylic acid methyl ester

The compound was obtained as described in EP 449196

### EXAMPLE 3

### 5,6-Dimethoxy-3-phenyl-1H-indole-2-carboxylic acid methyl ester

The compound was obtained as described in EP 449196.

The compounds described in Table 1 in examples 4-5 were synthesised following the process described in EP 449196 as for example 1.

### EXAMPLE 4

### 5,6-Dimethoxy-3-phenyl-1H-indole-2-carboxylic acid ethyl ester

TSQ 700, 400 uA, 70V, 50-300° C.; MS (m/z): 325 (M+)

¹H-NMR (CDCl₃) δ: 8.81 (s br, 1H); 7.55 (d, 2H); 7.46 (dd, 2H); 7.37 (dd, 1H); 6.96 (s, 1H); 6.87 (s, 1H); 4.26 (q, 2H); 3.96 (s, 3H); 3.85 (s, 3H); 1.22 (t, 3H).

### EXAMPLE 5

### 3-(4-Methoxyphenyl)-1H-indole-2-carboxylic acid methyl ester

ESI Pos, 3.5 KV, 20V, 300° C.; MS (m/z): 282.1 (MH+)

¹H-NMR (D₆₋DMSO) δ: 11.83 (s br, 1H); 7.49 (m, 2H); 7.42 (d, 2H); 7.29 (dd, 1H); 7.07 (dd, 1H); 7.02 (d, 2H); 3.82 (s, 3H); 3.76 (s, 3H).

### EXAMPLE 6

### 1-Benzyl-5,6-dimethoxy-3-(4-methoxyphenyl)-1H-indole-2-carboxylic acid methyl ester

80 mg (0.234) mmol of 5,6-dimethoxy-3-(4-methoxyphenyl)-1H-indole-2-carboxylic acid methyl ester are dissolved in 3 ml of anhydrous DMF under nitrogen. The solution is brought to 0° C. and 11.2 mg (0.281 mmol) of NaH (60% suspension in mineral oil) are added. The reaction mixture is maintained under stirring at ambient temperature for 1 hour.

33 μl (0.281 mmol) of benzyl bromide are added and the reaction mixture is left under stirring for 15 minutes. The mixture is diluted with Et₂O and the reaction is quenched with a saturated NH₄Cl solution. The organic phase is separated and the aqueous phase is extracted with Et₂O. The organic phases are combined, washed with NaClss, dried over anhydrous Na₂SO₄ and the solvent is evaporated under vacuum. The crude product obtained is purified by chromatography (eluent: AcOEt 1, petroleum ether 4). 70 mg of product are obtained. Yield: 69% ZQ, ESI POS, spray 3,25 KV/source 30 V/probe 250 C; MS (m/z): 432 (MH+)

¹H-NMR (CDCl₃) δ: 7.38 (d, 2H); 7.28 (dd, 2H); 7.22 (d, 1H); 7.10 (d, 2H); 7.00 (d, 2H); 6.92 (s, 1H); 6.74 (s, 1H); 5.77 (s, 2H); 3.89 (s, 3H); 3.87 (s, 3H); 3.84 (s, 3H); 3.61 (s, 3H).

The compounds described in Table 1 in examples 7-11 were synthesised following the process described in example 6.

### EXAMPLE 7

### 5,6-Dimethoxy-1-methoxycarbonylmethyl-3-(4-methoxyphenyl)-1H-indole-2-carboxylic acid methyl ester

ZQ, ESI POS, spray 3,25 KV/source 30 V/probe 250 C; MS (m/z): 414 (MH+)

¹H-NMR (CDCl₃) δ: 7.38 (d, 2H); 6.99 (d, 2H); 6.89 (s, 1H); 6.69 (s, 1H); 5.26 (s, 2H); 3.97 (s, 3H); 3.89 (s, 3H); 3.83 (s, 3H); 3.79 (s, 3H); 3.65 (s, 3H).

### EXAMPLE 8

### 1-Dimethylcarbamoylmethyl-5,6-dimethoxy-3-(4-methoxyphenyl)-1H-indole-2-carboxylic acid methyl ester

ZQ, ESI POS, spray 3,25 KV/source 30 V/probe 250 C; MS (m/z): 427 (MH+)

¹H-NMR (CDCl₃) δ: 7.37 (d, 2H); 6.97 (d, 2H); 6.87 (s, 1H); 6.70 (s, 1H); 5.36 (s, 2H); 3.96 (s, 3H); 3.88 (s, 3H); 3.82 (s, 3H); 3.61 (s, 3H); 3.20 (s br, 3H); 3.03 (s br, 3H).

### EXAMPLE 9

### 5,6-Dimethoxy-3-(4-methoxyphenyl)-1-propyl-1H-indole-2-carboxylic acid methyl ester

AQA, ESI POS, spray 3,25 KV/source 30 V/probe 250 C; MS (m/z): 384.1 (MH+)

¹H-NMR (CDCl₃) δ: 7.35 (d, 2H); 7.98 (d, 2H); 6.89 (s, 1H); 6.79 (s, 1H); 4.46 (dd, 2H); 3.99 (s, 3H); 3.88 (s, 3H); 3.83 (s, 3H); 3.66 (s, 3H); 1.86 (m, 2H); 0.98 (t, 3H).

### EXAMPLE 10

### 1-Cyanomethyl-5,6-dimethoxy-3-(4-methoxyphenyl)-1H-indole-2-carboxylic acid methyl ester

ESI Pos, 3.5 KV, 30V, 250° C.; MS (m/z): 381.0 (MH+)

¹H-NMR (CDCl₃) δ: 7.34 (d, 2H); 7.00 (d, 2H); 6.88 (s, 1H); 6.81 (s, 1H); 5.58 (s, 2H); 4.02 (s, 3H); 3.89 (s, 3H); 3.84 (s, 3H); 3.71 (s, 3H).

### EXAMPLE 11

### 1-(2-Dimethylaminoethyl)-5,6-dimethoxy-3-(4-methoxyphenyl)-1H-indole-2-carboxylic acid methyl ester hydrochloride

AQA, ESI POS, spray 3,25 KV/source 30 V/probe 250 C; MS (m/z): 413.1 (MH+)

¹H-NMR (CDCl₃) 6 (determined as base): 7.34 (d, 2H); 6.98 (d, 2H); 6.88 (s, 1H); 6.86 (s, 1H); 4.62 (m, 2H); 3.98 (s, 3H); 3.88 (s, 3H); 3.83 (s, 3H); 3.67 (s, 3H); 2.70 (m, 2H); 2.37 (s, 6H).

### EXAMPLE 12

### 1-(2-Hydroxyethyl)-5,6-dimethoxy-3-(4-methoxyphenyl)-1H-indole-2-carboxylic acid methyl ester

80 mg (0.234) mmol of 5,6-dimethoxy-3-(4-methoxyphenyl)-1H-indole-2-carboxylic acid methyl ester are dissolved in 3 ml of anhydrous DMF under nitrogen. The solution is brought to 0° C. and 11.2 mg (0.281 mmol) of NaH (60% suspension in mineral oil) are added. The reaction mixture is maintained under stirring at ambient temperature for 1 hour.

42 μl (0.281 mmol) of 2-(2-bromoethoxy)-tetrahydropyran are added and the mixture is left under stirring at ambient temperature for 24 hours. The mixture is diluted with Et₂O and the reaction is quenched with a saturated NH₄Cl solution. The organic phase is separated and the aqueous phase is extracted with Et₂O. The organic phases are combined, washed with NaClss, dried over anhydrous Na₂SO₄ and the solvent evaporated under vacuum. The crude product obtained is purified by chromatography (eluent: AcOEt 1, petroleum ether 1). 35 mg of product are obtained. Yield: 34%.

ZQ, ESI POS, spray 3,25 KV/source 30 V/probe 250 C; MS (m/z): 386 (MH+)

¹H-NMR (CDCl₃) δ: 7.34 (d, 2H); 6.99 (d, 2H); 6.87 (s, 1H); 6.86 (s, 1H); 4.65 (dd, 2H); 4.08 (dd, 2H); 3.98 (s, 3H); 3.89 (s, 3H); 3.83 (s, 3H); 3.66 (s, 3H).

### EXAMPLE 13

### 5,6-Dimethoxy-3-(4-methoxyphenyl)-1H-indole-2-carboxylic acid

The compound was obtained as described in EP 449196.

### EXAMPLE 14

### 5,6-Dimethoxy-3-(4-methoxyphenyl)-1H-indole-2-carboxyamide

690 mg (2.110 mmol) of 5,6-dimethoxy-3-(4-methoxyphenyl)-1H-indole-2-carboxylic acid are dissolved in 12.5 ml acetonitrile; 359 mg (2.215 mmol) of N,N′-carbonyldiimidazole are added and the mixture is refluxed for 2 hours while under stirring. The reaction mixture is cooled to 40° C., 30 ml of 30% NH₄OH are added and refluxed for 30 minutes. The solvent is evaporated under vacuum. The residue is re-dissolved with CH₂Cl₂, washed with H₂O and NaClss then dried over anhydrous Na₂SO₄. The solvent is evaporated under vacuum and 650 mg of crude product are obtained. The product is purified by means of a chromatographic column (eluent: AcOEt 2, CH₂Cl₂ 1). 580 mg of product are obtained. Yield: 84%.

API2000, ESI pos, Direct inj, DP30, FP 200, EP10, T350 C, gas 130, gas 2 50; MS (m/z): 327.3 (MH+)

¹H-NMR (CDCl₃) δ: 9.09 (s br, 1H); 7.44 (d, 2H); 7.08 (d, 2H); 6.88 (s, 1H); 7.77 (s, 1H); 5.74 (s br, 1H); 5.31 (s br, 1H); 3.96 (s, 3H); 3.90 (s, 3H); 3.83 (s, 3H).

### EXAMPLE 15

### 2-Amino-methyl-5,6-dimethoxy-3-(4-methoxyphenyl)-1H-indole hydrochloride

80 mg (0.245 mmol) of 5,6-dimethoxy-3-(4-methoxyphenyl)-1H-indole-2-carboxyamide are suspended in 3 ml of anhydrous THF under N₂. A 1 M solution of LiAlH₄ in THF (1.101 ml) is added drop-wise and the suspension is brought to 50° C. for 6 hours.

The reaction is quenched with H₂O and with 1 N NaOH, the precipitate obtained is filtered off then washed with Et₂O. The organic phase is washed with 0.1 N HCl, the aqueous phase is basified with 0.1 N NaOH and extracted with CH₂Cl₂. The organic phase is dried over Na₂SO₄ and the solvent evaporated under vacuum. The crude product obtained is purified by means of a chromatographic column (eluent: CH₂Cl₂ 20, MeOH 1.2, 30% NH₄OH 0.1). The product obtained is dissolved in Et₂O and treated with a solution of HCl in Et₂O. The precipitate is filtered and dried under vacuum. 32 mg of product are obtained. Yield: 37%.

ESI POS 3.5 kV, 30V, 250 C; MS (m/z): 296.3 ([MH+]-NH₃)

¹H-NMR (D₆₋DMSO) δ: 11.05 (s, 1H); 8.40 (s br, 3H); 7.44 (d, 2H); 7.08 (d, 2H); 7.02 (s, 1H); 6.95 (s, 1H); 4.13 (s, 2H); 3.83 (s, 3H); 3.82 (s, 3H); 3.70 (s, 3H).

### EXAMPLE 16

### N-[5,6-dimethoxy-3-(4-methoxyphenyl)-1H-indol-2-ylmethyl]-acetamide

60 mg (0.192 mmol) of 2-amino-methyl-5,6-dimethoxy-3-(4-methoxyphenyl)-1H-indole are dissolved in 3 ml of CH₂Cl₂. 40 μl (0.288 mmol) of triethylamine are added and the reaction mixture is brought to 0° C. 15 μl (1.1 mmol) of acetyl chloride are added and the temperature is brought to ambient. After 15 min the mixture is diluted with CH₂Cl₂, washed with a 5% citric acid solution, dried over Na₂SO₄ and the solvent evaporated under vacuum. The crude product obtained is purified by chromatography (eluent: CH₂Cl₂ 100, MeOH 3). 57 mg of product are obtained. Yield: 84%.

API 2000, ESI pos, direct inj, EP 30, FP 200, EP 10, T 350 C, gas 1 30, gas 2 50; MS (m/z): 355.4 (MH+)

¹H-NMR (CDCl₃) δ: 9.01 (s br, 1H); 7.34 (d br, 2H); 7.02 (d, 2H); 7.01 (s, 1H); 6.88 (s, 1H); 5.97 (t br, 1H); 4.48 (d br, 2H); 3.92 (s, 3H); 3.88 (s, 3H); 3.85 (s, 3H); 2.01 (s, 3H).

### EXAMPLE 17

### 5,6-dimethoxy-3-(4-methoxyphenyl)-1H-indole-2-carbonitrile

100 mg (0.306 mmol) of 5,6-dimethoxy-3-(4-methoxyphenyl)-1H-indole-2-carboxyamide are dissolved in a solution of dioxane (1 ml) and pyridine (1 ml). The solution is brought to 0° C. and 1 ml of trifluoroacetic anhydride are added drop-wise. The reaction mixture is maintained at 0° C. for 30 minutes. The reaction is quenched with H₂O and extracted with Et₂O; the organic phase is washed with 1 N HCl, 5% NaHCO₃ and NaClss then dried over anhydrous Na₂SO₄. The solvent is evaporated under vacuum. The crude product obtained is purified by means of a chromatograph column (eluent: AcOEt 3, petroleum ether 1). 50 mg of product are obtained. Yield: 53%.

ZQ, ESI POS, spray 3,25 KV/source 30 V/probe 250 C; MS (m/z): 309 (MH+)

¹H-NMR (CDCl₃) δ: 8.36 (s br, 1H); 7.62 (d, 2H); 7.14 (s, 1H); 7.07 (d, 2H); 6.85 (s, 1H); 3.95 (s, 3H); 3.90 (s, 3H); 3.89 (s, 3H).

### EXAMPLE 18

### 1-(2-Dimethylaminoethyl)-5,6-dimethoxy-3-(4-methoxyphenyl)-1H-indole-2-carbonitrile hydrochloride

80 mg (0.259 mmol) of 5,6-dimethoxy-3-(4-methoxyphenyl)-1H-indole-2-carbonitrile are dissolved in 3 ml of anhydrous DMF under nitrogen. The solution is brought to 0° C., 33 mg (0.829 mmol) of 60% NaH are added and left for 1 hour under stirring at ambient temperature. 56 mg (0.388 mmol) of 2-chloroethylamine hydrochloride are added and the mixture is left for 24 hours under stirring at ambient temperature. The reaction is quenched with NH₄Clss, the mixture is extracted with Et₂O, the organic phase is washed with NaClss and dried over anhydrous Na₂SO₄. The solvent is evaporated under vacuum. The crude product obtained is purified by chromatography (eluent CH₂Cl₂ 100, MeOH 4). The product is treated with Et₂O/HCl and filtered. 78 mg of product are obtained. Yield: 72%.

TSQ 700, 400 uA, 70V, 50-300° C.; MS (m/z): 370 (M+)

¹H-NMR (D₆₋DMSO) δ: 11.20 (s br, 1H); 7.61 (d, 2H); 7.49 (s, 1H); 7.14 (d, 2H); 7.10 (s, 1H); 4.76 (m, 2H); 3.94 (s, 3H); 3.84 (s, 3H); 3.76 (s, 3H); 3.45 (m, 2H); 2.84 (s, 6H).

### EXAMPLE 19

### N-[5,6-dimethoxy-3-(4-methoxyphenyl)-1H-indole-2-carbonyl]-methanesulfonamide

112 mg (0.342 mmol) of 5,6-dimethoxy-3-(4-methoxyphenyl)-1H-indole-2-carboxylic acid are dissolved in 6 ml of anhydrous CH₂Cl₂ under nitrogen. 58 mg (0.358 mmol) of N,N′-carbonyldiimidazole are added and the mixture is heated under reflux for 15 minutes. The temperature is brought to ambient and a solution of 49 mg (0.513 mmol) of methanesulfonamide and 66 μl (0.444 mmol) of DBU in 3 ml of anhydrous CH₂Cl₂ is added. The mixture is refluxed for 2 hours. A solution of 16 mg (0.171 mmol) of methanesulfonamide and 22 μl (0.148 mmol) of DBU in 1 ml of anhydrous CH₂Cl₂ is added and the mixture is heated under reflux for 1 hour. The mixture is diluted with CH₂Cl₂, washed with a solution of 5% citric acid, dried over anhydrous Na₂SO₄ and the solvent evaporated under vacuum. The crude product obtained is purified by means of a chromatographic column (eluent CH₂Cl₂ 100, MeOH 1, AcOH 0.05). 43 mg of product are obtained. Yield: 31%. API 2000, ESI pos, direct inj, DP 30, FP 200, EP 10, T 350 C, gas 1 30, gas 2 50; MS (m/z): 405.6 (MH+)

¹H-NMR (CDCl₃) δ: 9.45 (s br, 1H); 8.02 (s br, 1H); 7.44 (d, 2H); 7.13 (d, 2H); 6.95 (s, 1H); 6.74 (s, 1H); 3.97 (s, 3H); 3.91 (s, 3H); 3.83 (s, 3H); 3.33 (s, 3H).

### EXAMPLE 20

### [5,6-Dimethoxy-3-(4-methoxyphenyl)-1H-indol-2-yl]-methanol

1.29 g (3.783 mmol) of 5,6-dimethoxy-3-(4-methoxyphenyl)-1H-indole-2-carboxylic acid methyl ester are dissolved in 18 ml of anhydrous THF, under nitrogen. The solution is brought to 0° C. and 215 mg (5.673 mmol) of LiAlH₄ are added in portions. The mixture is brought to ambient temperature. After 1 hour a further 71 mg (1.891 mmol) of LiAlH₄ are added then it is left to react for 30 minutes when a further 71 mg of LiAlH₄ are added. After 30 minutes the reaction mixture is cooled to 0° C. 350 μl of H₂O, 1.225 ml of 15% NaOH, 350 μl of H₂O are slowly added and then left under stirring for 15 minutes. The precipitate is filtered off, the solution is washed with NaClss and the solvent evaporated under vacuum. The crude product is triturated in Et₂O and filtered. 1.070 g of product are obtained. Yield: 90%.

TSQ700, EI, 70V, 200 uA, 50-300 C; MS (m/z): 313 (M+)

¹H-NMR (D₆₋DMSO) δ: 10.88 (s, 1H); 7.42 (d, 2H); 7.02 (d, 2H); 7.00 (s, 1H); 6.92 (s, 1H); 5.16 (t br, 1H); 4.55 (d, 2H); 3.80 (s, 3H); 3.78 (s, 3H); 3.72 (s, 3H).

### EXAMPLE 21

### [5,6-Dimethoxy-3-(4-methoxyphenyl)-1H-indol-2-ylmethyl]-methylamine hydrochloride

1.015 g (3.243 mmol) of [5,6-dimethoxy-3-(4-methoxyphenyl)-1H-indol-2-yl]methanol are suspended in 45 ml of CH₂Cl₂. 3.31 g (32.43 mmol) of 85% MnO₂ are added and the mixture is left under vigorous stirring for 1.5 hours. The reaction mixture is filtered through celite and the solvent is evaporated under vacuum. The crude product obtained is triturated in Et₂O and filtered. 770 mg of 5,6-dimethoxy-3-(4-methoxy-phenyl)-1H-indole-2-carbaldehyde are obtained. Yield: 76%.

¹H-NMR: 300 MHz, DMSO, δ: 11.72 (s br, 1H); 9.60 (s, 1H); 7.55 (d, 2H); 7.11 (d, 2H); 7.02 (s, 1H); 6.91 (s, 1H); 3.83 (s, 6H); 3.75 (s, 3H). 100 mg (0.321 mmol) of 5,6-dimethoxy-3-(4-methoxyphenyl)-1H-indole-2-carbaldehyde are dissolved in 4 ml of CH₂Cl₂ and 1 ml of MeOH. 43 mg (0.642 mmol) of methylamine hydrochloride are added and the mixture is left under stirring for 2 hours at ambient temperature. 30 mg (0.481 mmol) of NaBH₃CN are added and the mixture is left under stirring for 20 hours. The reaction is quenched with H₂O, then the mixture is basified with 1 M NaOH, extracted with CH₂Cl₂ and dried over anhydrous Na₂SO₄. The solvent is evaporated under vacuum. The crude product obtained is purified by chromatography (eluent: CH₂Cl₂ 100, MeOH 5, 30% NH₄OH 0.5). The product is treated with Et₂O/HCl and filtered. 59 mg of product are obtained. Yield: 51%.

TSQ 700, E.I, 400 uA, 1000 eV, 70V, T 50-300 C; MS (m/z): 326.1 (M+)

¹H-NMR (D₆₋DMSO) δ: 11.14 (s, 1H); 9.16 (s br, 1H); 7.42 (d, 2H); 7.07 (d, 2H); 7.01 (s, 1H); 6.92 (s, 1H); 4.21 (s, 2H); 3.82 (s, 3H); 3.81 (s, 3H); 3.71 (s, 3H); 2.50 (s, 3H).

### EXAMPLE 22

### 3-[5,6-Dimethoxy-3-(4-methoxyphenyl)-1H-indol-2-yl]-propionic acid ethyl ester

232 μl (1.158 mmol) of triethyl phosphonoacetate are dissolved in 3 ml of anhydrous THF, under nitrogen. The solution is brought to 0° C., 48 mg (1.197 mmol) of 60% NaH are added and left under stirring for 15 minutes. 120 mg (0.386 mmol) of 5,6-dimethoxy-3-(4-methoxyphenyl)-1H-indole-2-carbaldehyde are added and the mixture is left at ambient temperature for 1 hour.

The reaction is quenched with H₂O, the mixture is extracted with AcOEt and dried with anhydrous Na₂SO₄. The solvent is evaporated under vacuum. The crude product obtained is purified by chromatography (eluent: CH₂Cl₂ 100, ACOEt 2.5, 30% NH₄OH 0.25). The product obtained is dissolved in 5 ml of absolute EtOH, 5 ml of AcOEt and a drop of pyridine. 12 mg of 10% Pd/C are added and hydrogenation is carried out at 30 psi for 1.5 hours. The catalyst is filtered off, the filtrate is dried, re-dissolved in AcOEt and washed with 0.1 N HCl and with 5% NaHCO₃. After drying over anhydrous Na₂SO₄ the solvent is evaporated under vacuum. The crude product is purified by chromatography (eluent: AcOEt 1, petroleum ether 3). 95 mg of product are obtained. Yield: 64%

TSQ 700, E.I; 400 uA, 70V, 180° C. source, probe 50-250° C.; MS (m/z): 383.1 (M+)

¹H-NMR (D₆₋DMSO) δ: 10.72 (s, 1H); 7.35 (d, 2H); 7.02 (d, 2H); 6.89 (s, 1H); 6.88 (s, 1H); 4.02 (q, 2H); 3.80 (s, 3H); 3.77 (s, 3H); 3.69 (s, 3H); 2.99 (m, 2H); 2.67 (m, 2H); 1.13 (t, 3H).

### EXAMPLE 23

### 5,6-Dimethoxy-3-(4-methoxyphenyl)-2-(2H-[1,2,4]triazol-3-yl)-1H-indole

200 mg (0.613 mmol) of 5,6-dimethoxy-3-(4-methoxyphenyl)-1H-indole-2-carboxyamide are suspended in 1.6 ml of N,N-dimethylformamide dimethyl acetal and heated at 120° C. for 1.5 hours, then dried under vacuum. 236 mg of 5,6-dimethoxy-3-(4-methoxy-phenyl)-1H-indole-2-carboxylic acid 1-dimethylamino-methylideneamide are obtained. 1 ml of acetic acid is placed in a flask, 14 μl (0.288 mmol) of hydrazine monohydrate and 100 mg (0.262 mmol) of 1-dimethylamino-methylideneamide of 5,6-dimethoxy-3-(4-methoxy-phenyl)-1H-indole-2-carboxylic acid are added. The mixture is heated at 90° C. for 15 minutes, diluted with CH₂Cl₂, washed with a 5% NaHCO₃ solution and dried over anhydrous Na₂SO₄. The solvent is evaporated under vacuum. The crude product obtained is purified by chromatography (eluent: CH₂Cl₂ 100, MeOH 2). 57 mg of product are obtained. Yield: 62%.

API 2000, ESI pos, direct inj, DP30, FP 200, EP 10, T350 C, gas 1 30, gas 2 60; MS (m/z): 351.2 (MH+)

¹H-NMR (CDCl₃) δ: 9.52 (s br, 1H); 7.95 (s, 1H); 7.48 (d, 2H); 7.11 (d, 2H); 6.92 (s, 1H); 6.87 (s, 1H); 3.96 (s, 3H); 3.92 (s, 3H); 3.86 (s, 3H).

### EXAMPLE 24

### 2-(4,5-Dihydro-1H-imidazol-2-yl)-5,6-dimethoxy-3-(4-methoxyphenyl)-1H-indole trifluoroacetate

2.25 ml of anhydrous toluene are placed under nitrogen and 586 μl of a 2M AlMe₃ solution in toluene are added; the solution obtained is brought to 0° C. 78 μl (1.172 mmol) of ethylene diamine are added. The solution is left under stirring for 1 hour at ambient temperature after which the temperature is brought to 0° C. 200 mg (0.586 mmol) of 5,6-dimethoxy-3-(4-methoxyphenyl)-1H-indole-2-carboxylic acid methyl ester are added, the reaction mixture is refluxed for 30 minutes and is then diluted with CH₂Cl₂. The reaction is quenched with a 5% K₂CO₃ solution, the phases are separated and the aqueous phase is extracted with CH₂Cl₂. The phases are combined, dried over anhydrous Na₂SO₄ and the solvent evaporated under vacuum. The crude product obtained is purified by preparative HPLC (column: Symmetry C18, 5 μM; eluentA: H₂O, 0.1% TFA, eluent B: CH₃CN, 0.1% TFA; gradient: 2 min 100% A, 18 min from 100% A to 100% B, 2 min 100% B). 18 mg of product are obtained. Yield: 7%.

ESI Pos, 3.5 KV, 30V, 250° C.; MS (m/z): 352.1 (MH+)

¹H-NMR (CDCl₃) δ: 12.67 (s br, 1H); 8.83 (s br, 2H); 7.39 (d, 2H); 7.11 (d, 2H); 7.02 (s, 1H); 6.63 (s, 1H); 3.98 (s, 3H); 3.93 (s, 3H); 3.92 (s, 4H); 3.81 (s, 3H).

### EXAMPLE 25

### 5,6-Dimethoxy-3-(4-methoxyphenyl)2-(1H-tetrazol-5-yl)-1H-indole

A mixture of 80 mg (0.260 mmol) 5,6-dimethoxy-3-(4-methoxyphenyl)-1H-indol-2-carbonitrile and 400 μl Bu₃SnN₃ are heated at 110° C. for 24 hours under stirring. Another 400 μl of Bu₃SnN₃ are added and the mixture is heated at 130° C. for 5 hours. The temperature is brought to ambient, 4 ml of a solution of 1 ml concentrated HCl and 3 ml MeOH are added; the mixture is left at ambient temperature for 24 hours. 10 ml of H₂O are added then the mixture is extracted with CH₂Cl₂. The organic phase is dried over anhydrous Na₂SO₄ and the solvent evaporated under vacuum. The crude product is purified by preparative HPLC (column: Symmetry C18, 5 μM; eluentA: H₂O 90, CH₃CN 10, TFA 0.05, eluent B: H₂O 10, CH₃CN 90, TFA 0.05; gradient: 3 min 5% B, 27 min from 5% B to 100% B, 4 min 100% B). 15 mg of product are obtained. Yield: 16%.

ESI Pos, 3.5 KV, 30V, 250° C.; MS (m/z): 352.1 (MH+)

¹H-NMR (D₆₋DMSO) δ: 11.60 (s br, 1H); 7.36 (d, 2H); 7.01 (d, 2H); 6.99 (s, 1H); 6.98 (s, 1H); 3.82 (s, 3H); 3.81 (s, 3H); 3.74 (s, 3H).

### EXAMPLE 26

### 5,6-Dimethoxy-3-(4-methoxyphenyl)-2-[1,3,4]oxadiazol-2-yl-1H-indole

200 mg (0.612 mmol) of 5,6-dimethoxy-3-(4-methoxyphenyl)-1H-indole-2-carboxylic acid are dissolved in 10 ml of anhydrous CH₂Cl₂ under nitrogen. 104 mg (0.641 mmol) of N,N′-carbonyldiimidazole are added and the mixture is refluxed for 30 minutes. The reaction mixture is brought to ambient temperature, 60 μl (1.224 mmol) of hydrazine monohydrate are added and then left under stirring for 30 minutes.

The mixture is diluted with CH₂Cl₂ and washed with H₂O. The phases are separated and the aqueous phase is extracted with CH₂Cl₂. The organic phases are combined, dried over anhydrous Na₂SO₄ and the solvent evaporated under vacuum. The crude product obtained is purified by chromatography (eluent: CH₂Cl₂ 20, MeOH 1).

197 mg of product are obtained. Yield: 94%.

¹H-NMR (D₆₋DMSO) δ: 11.28 (s br, 1H); 8.12 (s br, 1H); 7.40 (d, 2H); 7.04 (d, 2H); 6.92 s, 1H); 6.81 (s, 1H); 4.36 (d br, 2H); 3.82 (s, 3H); 3.80 (s, 3H); 3.69 (s, 3H). A mixture of 120 mg (0.352 mmol) 5,6-dimethoxy-3-(4-methoxyphenyl)-1H-indole-2-carboxyhydrazide and 900 μl formic acid is heated at 80° C. for 30 minutes. The formic acid is evaporated under vacuum, the crude product is taken up in CH₂Cl₂, petroleum ether is added and the precipitate filtered. 90 mg of a white solid are obtained. A mixture of 90 mg of the solid obtained and 375 μl POCl₃ is heated at 80° C. for 15 minutes. The POCl₃ is evaporated, the remainder is taken up in CH₂Cl₂ and washed with H₂O; the aqueous phase is extracted with CH₂Cl₂. The organic phases are combined, dried over anhydrous Na₂SO₄ and the solvent evaporated under vacuum. The crude product obtained is purified by chromatography (eluent: CH₂Cl₂ 100, MeOH 1).

54 mg of product are obtained. Yield: 44%.

ESI Pos, 3.5 KV, 20V, 300° C.; MS (m/z): 352.1 (MH+)

¹H-NMR (D₆₋DMSO) δ: 11.90 (s br, 1H); 9.15 (s, 1H); 7.47 (d, 2H); 7.05 (d, 2H); 6.98 (s, 1H); 6.90 (s, 1H); 3.83 (s, 6H); 3.73 (s, 3H).

### EXAMPLE 27

### 7,8-Dimethoxy-10-(4-methoxyphenyl)-3,4-dihydro-2H-pyrazino[1,2-a]indol-1-one

250 mg (0.657 mmol) of 1-cyanomethyl-5,6-dimethoxy-3-(4-methoxyphenyl)-1H-indole-2-carboxylic acid methyl ester are suspended in 10 ml of absolute EtOH. 246 μl of 12% HCl and 20 mg of 10% Pd/C are added and the mixture is hydrogenated at 43 psi for 24 hours. 15 ml of AcOEt are added to better solubilize the starting product, a further 246 μl of 12% HCl are added then a further 20 mg of 10% Pd/C. The mixture is hydrogenated at 43 psi for 24 hours. A further 15 mg of 10% Pd/C are added and the mixture is hydrogenated for 24 hours. The mixture is filtered over paper and the solvent evaporated under vacuum. 242 mg of 1-(2-amino-ethyl)-5,6-dimethoxy-3-(4-methoxyphenyl)-1H-indole-2-carboxylic acid methyl ester hydrochloride are obtained. Yield: 87%. 170 mg (0.404 mmol) of 1-(2-amino-ethyl)-5,6-dimethoxy-3-(4-methoxyphenyl)-1H -indole-2-carboxylic acid methyl ester hydrochloride are dissolved in H₂O. The mixture is basified with 5% K₂CO₃, extracted with CH₂Cl₂, dried over anhydrous Na₂SO₄ and the solvent evaporated under vacuum. The product obtained is dissolved in 10 ml anhydrous toluene, 303 μl of 2M AlMe₃ in toluene are added and the mixture is heated at 80° C. for 10 minutes. It is brought to ambient temperature, the reaction is quenched with H₂O and the mixture extracted with AcOEt. The organic phase is washed with NaClss, dried over anhydrous Na₂SO₄ and the solvent evaporated under vacuum. The crude product obtained is purified by chromatography (eluent: CH₂Cl₂ 100, MeOH 1.5). 91 mg of product are obtained. Yield: 64%.

TSQ 700, EI, 200 uA, 70V, 50-300 C; MS (m/z): 352.1 (M+)

¹H-NMR (D₆₋DMSO) δ: 7.81 (t br, 1H); 7.48 (d, 2H); 7.13 (s, 1H); 6.97 (d, 2H); 6.93 (s, 1H); 4.25 (m, 2H); 3.86 (s, 3H); 3.81 (s, 3H); 3.72 (s, 3H); 3.61 (m, 2H).

### EXAMPLE 28

### 7,8-Dimethoxy-10-(4-methoxyphenyl)-1,2,3,4-tetrahydro-pyrazino[1,2-a]indole hydrochloride

60 mg (0.170 mmol) of 7,8-dimethoxy-10-(4-methoxyphenyl)-3,4-dihydro-2H-pyrazino[1,2-a]indol-1-one are suspended in 2.5 ml of anhydrous THF, under nitrogen. The mixture is brought to 0° C. and 22 mg (0.595 mmol) of LiAlH₄ are added. The reaction mixture is heated at 60° C. for 4 hours. The reaction is quenched with H₂O and 1M NaOH. The precipitate is filtered off, the solution dried over anhydrous Na₂SO₄ and the solvent evaporated under vacuum. The crude product obtained is purified by chromatography (eluent: CH₂Cl₂ 100, MeOH 2). The product obtained is treated with Et₂O/HCl and filtered. 30 mg of product are obtained. Yield: 47%.

TSQ 700, EI, 200 uA, 70V, source 180 C, MS (m/z): 338.3 (M+)

¹H-NMR (D₆₋DMSO) δ: 9.50 (s br, 1H); 7.37 (d, 2H); 7.13 (s, 1H); 7.07 (d, 2H); 7.07 (s, 1H); 4.50 (s, 2H); 4.28 (m, 2H); 3.84 (s, 3H); 3.81 (s, 3H); 3.75 (s, 3H); 3.69 (m, 2H).

### EXAMPLE 29

### 5,6-Dimethoxy-3-phenyl-1H-indole-2-carboxylic acid

Prepared following process described in EP 449196 EI, 70 eV, 150° C., 50-300° C.; MS (m/z): 297.1 (M⁺)

¹H-NMR (CDCl₃): δ 8.99 (s br, 1H); 7.57 (dd, 2H); 7.47 (dd, 2H); 7.39 (dd, 1H); 7.35 (s br, 1H); 6.94 (s, 1H); 6.82 (s, 1H); 3.93 (s, 3H); 3.84 (s, 3H).

### EXAMPLE 30

### 3-(4-Chlorophenyl)-5,6-dimethoxy-1H-ndole-2-carboxylic acid methyl ester

Prepared following process described in EP 449196

ESI POS, 3.5 KV, 20V, 300° C.; MS (m/z): 346.2 (MH⁺)

¹H-NMR (CDCl₃): δ 8.82 (s, 1H), 7.49-7.43 (m, 4H), 6.90 (s, 1H), 6.86 (s, 1H), 3.96 (s, 3H), 3.85 (s, 3H), 3.80 (s, 3H).

### EXAMPLE 31

### 3-(4-(Trifluoromethyl)phenyl)-5,6-dimethoxy-1H-indole-2-carboxylic acid methyl ester

Prepared following process described in EP 449196

ESI POS, 3.5 KV, 20V, 300° C.; MS (m/z): 380.2 (MH⁺)

¹H-NMR (CDCl₃) δ 8.95 (s, 1H), 7.74 (d, 2H), 7.67 (d, 2H), 6.90 (s, 1H), 6.89 (s, 1H), 3.98 (s, 3H), 3.88 (s, 3H), 3.82 (s, 3H).

### EXAMPLE 32

### 5,6-Dimethoxy-3-p-tolyl-1H-indole-2-carboxylic acid methyl ester

Prepared following process described in EP 449196

ESI POS, 3.5 KV, 20V, 300° C.; MS (m/z): 326.3 (MH⁺)

¹H-NMR (CDCl₃) δ 8.92 (s, 1H), 7.47 (d, 2H), 7.30 (d, 2H), 6.97 (s, 1H), 6.86 (s, 1H), 3.96 (s, 3H), 3.86 (s, 3H), 3.80 (s, 3H), 2.45 (s, 3H).

### EXAMPLE 33

### 3-(4-Fluorophenyl)-5,6-dimethoxy-1H-indole-2-carboxylic acid methyl ester

Prepared following process described in EP 449196

ESI POS, 3.5 KV, 20V, 300° C.; MS (m/z): 330.2 (MH⁺)

¹H-NMR (CDCl₃) δ (ppm): 8.94 (s, 1H), 7.52 (dd, 2H), 7.18 (dd, 2H), 6.91 (s, 1H), 6.88 (s, 1H), 3.97 (s, 3H), 3.87 (s, 3H), 3.81 (s, 3H).

### EXAMPLE 34

### 3-(2-Chlorophenyl)-5,6-dimethoxy-1H-indole-2-carboxylic acid methyl ester

Prepared following process described in EP 449196

ESI POS, 3.5 KV, 20V, 300° C.; MS (m/z): 346.2 (MH⁺)

¹H-NMR (CDCl₃) δ (ppm): 8.87 (s, 1H), 7.55-7.51 (m, 1H), 7.43-7.38 (m, 1H), 7.37-7.32 (m, 2H), 6.88 (s, 1H), 6.71 (s, 1H), 3.96 (s, 3H), 3.83 (s, 3H), 3.75 (s, 3H).

### EXAMPLE 35

### 3-(3-Chlorophenyl)-5,6-dimethoxy-1H-indole-2-carboxylic acid methyl ester

Prepared following process described in EP 449196

ESI POS, 3.5 KV, 20V, 300° C.; MS (m/z): 346.2 (MH⁺)

¹H-NMR (CDCl₃) δ (ppm): 8.91 (s, 1H), 7.54 (s, 1H), 7.43-7.37 (m, 3H), 6.91 (s, 1H), 6.87 (s, 1H), 3.96 (s, 3H), 3.86 (s, 3H), 3.80 (s, 3H).

### EXAMPLE 36

### 5-Chloro-3-phenyl-1H-indole-2-carboxylic acid ethyl ester

Prepared following process described in EP 449196

ESI POS, 3.2 KV, 20V, 300° C.; MS (m/z): 300.1 (MH⁺)

¹H-NMR (CDCl₃) δ (ppm): 8.98 (s br, 1H); 7.60-7.27 (m, BH); 4.29 (q, 2H); 1.23 (t, 3H).

### EXAMPLE 37

### 5-Fluoro-3-phenyl-1H-indole-2-carboxylic acid ethyl ester

Prepared following process described in EP 449196

ESI POS, 3.2 KV, 20V, 300° C.; MS (m/z): 284.1 (MH⁺)

¹H-NMR (CDCl₃) δ (ppm): 8.97 (s br, 1H); 7.58-7.51 (m, 6H); 7.26 (dd, 1H); 7.11 (ddd, 1H); 4.28 (q, 2H); 1.23 (t, 3H).

### EXAMPLE 38

### 5-Methoxy-3-phenyl-1H-indole-2-carboxylic acid ethyl ester

Prepared following process described in EP 449196

ESI POS, 3.2 KV, 20V, 300° C.; MS (m/z): 296.2 (MH⁺)

¹H-NMR (CDCl₃) δ (ppm): 8.86 (s br, 1H); 7.55 (dd, 2H); 7.46 (dd, 2H); 7.40 (dd, 1H); 7.34 (d, 1H); 7.03 (dd, 1H); 7.00 (d, 1H); 4.28 (q, 2H); 3.79 (s, 3H); 1.22 (t, 3H).

### EXAMPLE 39

### 5,6-Dimethoxy-1-(2-methoxyethyl)-3-(4-methoxyphenyl)-1H-indole-2-carboxylic acid methyl ester

Prepared following process described in Example 6

ESI Pos, 3.2 KV, 20V, 300° C.; MS (m/z): 400.1 (MH+)

¹H-NMR (CDCl₃) δ (ppm): 7.35 (d, 2H); 6.99 (d, 2H); 6.94 (s, 1H); 6.86 (s, 1H); 4.66 (t, 2H); 3.98 (s, 3H); 3.89 (s, 3H); 3.83 (s, 3H); 3.79 (t, 2H); 3.66 (s, 3H); 3.34 (s, 3H).

### EXAMPLE 40

### 1-(2-Hydroxyethyl)-5,6-dimethoxy-3-phenyl-1H-indole-2-carbonitrile

Prepared following process described in Example 12

ESI POS, spray 3 KV, 20V, 300° C.; MS (m/z): 323.3 (MH⁺)

¹H-NMR (CDCl₃) δ (ppm): 7.90 (d, 2H), 7.76 (m, 3H), 7.64 (m, 1H), 7.38 (s, 1H), 7.12 (s, 1H), 4.67 (m, 2H), 4.36-4.27 (m, 3H), 4.21 (s, 3H), 4.12 (s, 3H).

### EXAMPLE 41

### 1-(3-Hydroxypropyl)-5,6-dimethoxy-3-(4-methoxyphenyl)-1H-indole-2-carboxylic acid methyl ester

Prepared following process described in Example 12

ESI POS, spray 3 KV, 20V, 300° C.; MS (m/z): 400.3 (MH⁺)

¹H-NMR (CDCl₃) δ (ppm): 7.33 (d, 2H), 6.99 (d, 2H), 6.88 (s, 2H), 4.64 (t, 2H), 3.98 (s, 3H), 3.89 (s, 3H), 3,83 (s, 3H), 3,65 (s, 3H), 3,62 (t, 2H), 2.12 (m, 2H).

### EXAMPLE 42

### 1-(2-Hydroxyethyl)-5,6-dimethoxy-3-(4-methoxyphenyl)-1H-indole-2-carbonitrile

Prepared following process described in Example 12

ESI POS, spray 3 KV, 20V, 300° C.; MS (m/z): 353.3 (MH⁺)

¹H-NMR (CDCl₃) δ (ppm): 7.62 (d, 2H), 7.13 (s, 1H), 7.07 (d, 2H), 6.88 (s, 1H), 4.43 (t, 2H), 4.08 (m, 2H), 3.98 (s, 3H), 3.90 (s, 3H), 3.88 (s, 3H).

### EXAMPLE 43

### 2-(5,6-Dimethoxy-3-(4-methoxyphenyl)-2-(5-methyl-1,3,4-oxadiazol-2-yl)-1H-indol-1-yl)ethanol

Prepared following process described in Example 12

ESI POS, spray 3 KV, 20V, 300° C.; MS (m/z): 410.4 (MH⁺)

¹H-NMR (CDCl₃) δ (ppm): 7.35 (d, 2H), 6.99 (d, 2H), 6,96 (s, 1H), 6.88 (s, 1H), 4.71 (t, 2H), 4.14 (t, 2H), 3.99 (s, 3H), 3.89 (s, 3H), 3.86 (s, 3H), 2.40 (s, 3H).

### EXAMPLE 44

### 1-(3-Hydroxypropyl)-5,6-dimethoxy-3-(4-methoxyphenyl)-1H-indole-2-carbonitrile

Prepared following process described in Example 12

ESI POS, 3.5 KV, 20V, 3000° C.; MS (m/z): 367.2 (MH⁺).

¹H-NMR (CDCl₃): δ 7.62 (d, 2H), 7.13 (s, 1H), 7.08 (d, 2H), 6.91 (s, 1H), 4.45 (t, 2H), 4.00 (s, 3H), 3.92 (s, 3H), 3.89 (s, 3H), 3.70 (t, 2H), 2.18-2.11 (m, 2H).

### EXAMPLE 45

### 5,6-Dimethoxy-3-phenyl-1H-indole-2-carboxylic acid amide

Prepared following process described in Example 14

ESI POS, spray 3.5 KV, source 20V, probe 300° C.; MS (m/z): 297.3 (MH⁺)

¹H-NMR (CDCl₃) δ (ppm): 9.10 (s, 1H), 7.60-7.40 (m, 5H), 6.88 (s, 1H), 6.78 (s, 1H), 5.73 (b s, 1H), 5.24 (b s, 1H), 3.96 (s, 3H), 3.82 (s, 3H).

### EXAMPLE 46

### 5,6-Dimethoxy-3-phenyl-1H-indole-2-carboxylic acid dimethylamide

Prepared following process described in Example 14

ESI Pos, 3.2 KV, 20V, 300° C.; MS (m/z): 325.2 (MH⁺).

¹H-NMR (CDCl₃) δ (ppm): 8.94 (s br, 1H); 7.50-7.42 (m, 4H); 7.35 (m, 1H); 7.14 (s, 1H); 6.91 (s, 1H); 3.93 (s, 3H); 3.89 (s, 3H); 2.98-2.49 (s br, 6H).

### EXAMPLE 47

### (5,6-Dimethoxy-3-phenyl-1H-indol-2-yl)-morpholin-4-yl-methanone

Prepared following process described in Example 14

ESI Pos, 3.2 KV, 20V, 300° C.; MS (m/z): 367.1 (MH⁺).

¹H-NMR (CDCl₃) δ (ppm): 8.79 (s br, 1H); 7.53-7.44 (m, 4H); 7.44-7.34 (m, 1H); 7.09 (s, 1H); 6.90 (s, 1H); 3.94 (s, 3H); 3.88 (s, 3H); 3.37 (m br, 4H); 3.23 (m br, 4H).

### EXAMPLE 48

### 5,6-Dimethoxy-3-phenyl-1H-indole-2-carboxylic acid methylamide

Prepared following process described in Example 14

ESI Pos, 3.2 KV, 20V, 300° C.; MS (m/z): 311.1 (MH⁺).

¹H-NMR (CDCl₃) δ (ppm): 9.26 (s br, 1H); 7.60-7.43 (m, 5H); 6.91 (s, 1H); 6.79 (s, 1H); 5.80 (m br, 1H); 3.95 (s, 3H); 3.82 (s, 3H); 2.81 (d, 3H).

### EXAMPLE 49

### 5,6-Dimethoxy-3-phenyl-1H-indole-2-carbonitrile

Prepared following process described in Example 17

ESI POS, spray 3.5 KV, source 20V, probe 300° C.; MS (m/z): 279.2 (MH⁺)

¹H-NMR (CDCl₃): δ 8.37 (s, 1H), 7.70 (d, 2H), 7.54 (t, 2H), 7.43 (t, 1H), 7.16 (s, 1H), 6.87 (s, 1H), 3.97 (s, 3H), 3.91 (s, 3H).

### EXAMPLE 50

### 5,6-Dimethoxy-3-phenyl-1-propyl-1H-indole-2-carbonitrile

Prepared following process described in Example 18

ESI POS, spray 3 KV, 20V, 300° C.; MS (m/z): 321.3 (MH⁺)

¹H-NMR (CDCl₃) δ (ppm): 7.68 (d, 2H), 7.52 (t, 2H) 7.39 (m, 2H), 7.17 (s, 1H), 6.75 (s, 1H), 4.25 (t, 2H), 3.99 (s, 3H), 3.91 (s, 3H), 1.97-1.91 (m, 2H), 1.54 (s, 6H), 1.01 (m, 3H).

### EXAMPLE 51

### 1-(2-(Dimethylamino)ethyl)-5,6-dimethoxy-3-phenyl-1H-indole-2-carbonitrile hydrochloride

Prepared following process described in Example 18

ESI POS, spray 3 KV, 20V, 300° C.; MS (m/z): 350.3 (MH⁺)

¹H-NMR (DMSO-d₆) δ (ppm): 10.95 (s, 1H), 7.67 (d, 2H), 7.56 (t, 2H) 7.45 (m, 2H), 7.11 (s, 1H), 4.77 (t, 2H), 3.93 (s, 3H), 3.77 (s, 3H), 3.48 (m, 2H), 2.86 (d, 6H).

### EXAMPLE 52

### 5,6-Dimethoxy-3-(4-methoxyphenyl)-1-(2-(pyrrolidin-1-yl)ethyl)-1H-indole-2-carboxylic acid methyl ester hydrochloride

Prepared following process described in Example 18

ESI POS, spray 3 KV, 20V, 300° C.; MS (m/z): 439.3 (MH⁺)

¹H-NMR (DMSO-d₆) δ (ppm): 11.06 (s, 1H), 7.38 (s, 1H), 7.29 (d, 2H) 6.99 (d, 2H), 6.78 (s, 1H), 4.85 (m, 2H), 3.92 (s, 3H), 3.80 (s, 3H), 3.67 (s, 3H) 3.61 (s, 3H), 3.60-3.45 (m, 4H), 3.01 (m, 2H), 2.06-1.80 (m, 4H).

### EXAMPLE 53

### 1-(3-(Dimethylamino)propyl)-5,6-dimethoxy-3-(4-methoxyphenyl)-1H-indole-2-carboxylic acid methyl ester hydrochloride

Prepared following process described in Example 18

ESI POS, spray 3 KV, 20V, 300° C.; MS (m/z): 427.3 (MH⁺)

¹H-NMR (DMSO-d₆) δ (ppm): 10.27 (s, 1H), 7.28 (d, 2H), 7.20 (s, 1H) 6.98 (d, 2H), 6.78 (s, 1H), 4.55 (t, 2H), 3.88 (s, 3H), 3.85 (s, 3H), 3.82 (s, 3H) 3.79 (s, 3H), 3.12 (m, 2H), 2.74 (s, 3H), 2.72 (s, 3H), 2.12 (m, 2H).

### EXAMPLE 54

### 5,6-Dimethoxy-3-(4-methoxyphenyl)-1-(2-(pyrrolidin-1-yl)ethyl)-1H-indole-2-carbonitrile hydrochloride

Prepared following process described in Example 18

ESI POS, spray 3 KV, 20V, 300° C.; MS (m/z): 406.3 (MH⁺)

¹H-NMR (DMSO-d₆) δ (ppm): 10.72 (s, 1H), 7.62 (d, 2H), 7.38 (s, 1H) 7.14 (d, 2H), 7.11 (s, 1H), 4.73 (t, 2H), 3.93 (s, 3H), 3.84 (s, 3H), 3.79 (s, 3H), 3.61 (m, 4H), 3.08 (m, 2H), 2.03 (m, 2H), 1.87 (m, 2H).

### EXAMPLE 55

### 1-(3-(Dimethylamino)propyl)-5,6-dimethoxy-3-(4-methoxyphenyl)-1H-indole-2-carbonitrile hydrochloride

Prepared following process described in Example 18 ESI POS, spray 3 KV, 20V, 300° C.; MS (m/z): 394.3 (MH⁺)

¹H-NMR (DMSO-d₆) δ (ppm): 7.61 (d, 2H), 7.23 (s, 1H) 7.13 (d, 2H), 7.10 (s, 1H), 4.40 (t, 2H), 3.90 (s, 3H), 3.84 (s, 3H), 3.78 (s, 3H), 3.29 (s, BH), 2.15 (m, 2H).

### EXAMPLE 56

### 5,6-Dimethoxy-3-(4-methoxyphenyl)-1-(2-morpholinoethyl)-1H-indole-2-carbonitrile hydrochloride

Prepared following process described in Example 18

ESI POS, spray 3 KV, 20V, 300° C.; MS (m/z): 422.3 (MH⁺)

¹H-NMR (DMSO-d₆) δ (ppm): 12.26 (s, 1H), 7.61 (d, 3H), 7.14 (d, 2H), 7.10 (s, 1H), 4.85 (t b, 2H), 3.94 (s, 3H), 3.83 (s, 3H), 3.78 (s, 3H), 3.50 (m, 4H), 3.07 (s, 4H).

### EXAMPLE 57

### 2-(5,6-Dimethoxy-3-(4-methoxyphenyl)-2-(5-methyl-1,3,4-oxadiazol-2-yl)-1H-indol-1-yl)-N,N-dimethylethanamine

Prepared following process described in Example 18

ESI POS, spray 3 KV, 20V, 300° C.; MS (m/z): 437.5 (MH⁺)

¹H-NMR (CDCl₃) δ (ppm): 7.34 (d, 2H), 6.97 (m, 4H), 4.76 (t b, 2H), 4.01 (s, 3H), 3.88 (s, 3H), 3.86 (s, 3H), 2.79 (t, 2H), 2.40 (s b, 9H).

### EXAMPLE 58

### 5,6-Dimethoxy-3-phenyl-2-(4H-1,2,4-triazol-3-yl)-1H-indole

Prepared following process described in Example 23

¹H-NMR (CDCl₃): δ 9.77 (s, 1H), 8.00 (s, 1H), 7.62-7.59 (m, 5H), 6.96 (s, 1H), 6.88 (s, 1H), 3.99 (s, 3H), 3.87 (s, 3H).

ESI POS, 3.5 KV, 20V, 300° C.; MS (m/z): 321.3 (MH⁺), 641(2 MH⁺).

### EXAMPLE 59

### 3,4-Dihydro-7,8-dimethoxy-10-phenylpyrazino[1,2-a]indol-1(2H)-one

Prepared following process described in Example 27

ESI POS, spray 3 KV, 20V, 300° C.; MS (m/z): 323.3 (MH⁺)

¹H-NMR (DMSO-d₆) δ (ppm): 7.88 (s, 1H), 7.54 (d, 2H), 7.40 (t, 2H), 7.29 (t, 1H), 7.15 (s, 1H), 6.93 (s, 1H), 4.27 (t, 2H), 3.86 (s, 3H), 3.71 (s, 3H), 3.62 (bt, 2H)

### EXAMPLE 60

### 1-(2-Aminoethyl)-5,6-dimethoxy-3-(4-methoxy-phenyl)-1H-indole-2-carboxylic acid methyl ester hydrochloride

The title compound was obtained as described in Example 27 and purified by trituration using a mixture of diethyl ether/dichloromethane (9.4 mg, 10% yield).

ESI POS, spray 3 KV, 20V, 300° C.; MS (m/z): 385.3 (MH⁺)

¹H-NMR (DMSO-d₆) δ (ppm): 8.25 (s, 1H), 7.52 (d, 3H), 7.51 (s, 1H), 7.21 (d, 2H), 7.01 (s, 1H), 4.90 (t, 2H), 4.11 (s, 3H), 4.02 (s, 3H), 3.90 (s, 3H), 3.82 (s, 3H), 3.44 (t, 2H).

### EXAMPLE 61

### 7,8-Dimethoxy-10-phenyl-1,2,3,4-tetrahydropyrazino[1,2-a]indole hydrochloride

Prepared following process described in Example 28

ESI POS, 3.5 KV, 20V, 300° C.; MS (m/z): 309.2 (MH⁺)

¹H-NMR (DMSO-d₆) δ (ppm): 9.10 (b s, 1H), 7.52-7.43 (m, 4H), 7.36-7.29 (m, 1H), 7.15 (s, 1H), 7.13 (s, 1H), 4.52 (s, 2H), 4.27 (t, 2H), 3.85 (s, 3H), 3.76 (s, 3H), 3.68 (t, 2H).

### EXAMPLE 62

### 5,6-Dimethoxy-3-(4-methoxyphenyl)-1-[2-(4-methylpiperazin-1-yl)-ethyl]-1H-indole-2-carboxylic acid methyl ester dihydrochloride

To a solution of 5,6-dimethoxy-3-(4-methoxyphenyl)-1-(2-hydroxyethyl)-1H-indole-2-carboxylic acid methyl ester (51.7 mg, 0.13 mmol) and 4-(N,N-dimethylaminopyridine) (19.5 mg, 0.16 mmol) in 5 ml of dichloromethane, tosyl chloride (30.6 mg, 0.16 mmol) was added at 0° C. The mixture was stirred at room temperature for 3 hours. The solvent was evaporated under vacuum and the crude residue was purified by silica gel column chromatography (CH₂Cl₂/MeOH, 99:1) to yield 54 mg of 5,6-dimethoxy-3-(4-methoxyphenyl)-1-[2-(toluene-4-sulfonyloxy)-ethyl]-1H-indole-2-carboxylic acid methyl ester (75% yield) as a yellowish solid.

ESI POS, spray 3 KV, cono 20V, 300° C.; MS (m/z): 540.3 (MH⁺)

To a solution of 5,6-dimethoxy-3-(4-methoxyphenyl)-1-[2-(toluene-4-sulfonyloxy)-ethyl]-1H-indole-2-carboxylic acid methyl ester (54 mg, 0.1 mmol) in 3 ml of dry acetonitrile, 1-methyl-piperazine (444 μl, 4.0 mmol) was added and the resulting mixture was stirred overnight at 65° C. After solvent evaporation under reduced pressure, the residue was dissolved in dichloromethane and washed sequentially with saturated aqueous NH₄Cl and brine. The organic phase was dried over Na₂SO₄ and dichloromethane was removed under reduced pressure. The residue was purified by flash chromatography (CH₂Cl₂/MeOH, 98:2) to give a colourless oil that was dissolved in diethyl ether and transformed into the correspondent hydrochloride adding few drops of concentrated HCl. The white solid was filtered, washed with diethyl ether and dried to yield the title compound (26 mg, 48% yield).

ESI POS, spray 3 KV, 20V, 300° C.; MS (m/z): 468 (MH⁺)

¹H-NMR (DMSO-d₆) δ (ppm): 7.31 (d, 3H), 7.01 (d, 2H), 6.80 (s, 1H), 4.81 (b s, 2H), 3.91 (s, 3H), 3.69 (s, 3H), 3.63 (s, 3H), 3.38 (b s 12H), 2.81 (b s, 3H).

### EXAMPLE 63

### 5,6-Dimethoxy-3-(4-methoxyphenyl)-1-(2-morpholinoethyl)-1H-indole-2-carboxylic acid methyl ester hydrochloride

Prepared following process described in Example 62

ESI POS, spray 3 KV, 20V, 300° C.; MS (m/z): 455.3 (MH⁺)

¹H-NMR (DMSO-d₆) δ (ppm): 11.4 (s, 1H), 7.42 (s, 1H), 7.30 (d, 2H), 7.00 (d, 2H), 6.79 (s, 1H), 5.01-4.88 (m, 2H), 4.06-3.16 (m, 10H,), 3.93 (s, 3H), 3.81 (s, 3H), 3.69 (s, 3H) 3.64 (s, 3H).

### EXAMPLE 64

### 5,6-Dimethoxy-3-(4-methoxyphenyl)-1-(2-(4-methylpiperazin-1-yl)ethyl)-1H-indole-2-carbonitrile dihydrochloride

Prepared following process described in Example 62

ESI POS, spray 3 KV, 20V, 300° C.; MS (m/z): 435.3 (MH⁺)

¹H-NMR (DMSO-d₆) δ (ppm): 7.61 (d, 2H), 7.21 (s, 1H), 7.13 (d, 2H), 7.08 (s, 1H), 4.42 (b t, 2H), 3.89 (s, 3H), 3.83 (s, 3H), 3.77 (s, 3H), 3.02 (d, 2H), 2.93 (d, 2H), 2.74 (m, 5H), 2.51-2.32 (4H, m).

### EXAMPLE 65

### 7,8-Dimethoxy-10-(4-methoxyphenyl)-2-methyl-1,2,3,4-tetrahydro-pyrazino[1,2-a]indole hydrocloride

To a solution of 5,6-dimethoxy-3-(4-methoxyphenyl)-1-(2-hydroxyethyl)-1H-indole-2-carboxylic acid methyl ester (195 mg, 0.50 mmol) and diisopropylethylamine (351 μl, 2.01 mmol) in dry THF (8 ml), methanesulfonic anhydride (174.2 mg, 1.0 mmol) in 2 ml of dry THF was added dropwise at 0° C. The solution was stirred for 2 hours at r.t. After evaporation of the solvent, the residue was dissolved in dichloromethane, washed with diluted aqueous NH₄Cl, dried over Na₂SO₄ and concentrated. The resulting crude 1-(2-methanesulfonyloxy-ethyl)-5,6-dimethoxy-3-(4-methoxy-phenyl)-1H-indole-2-carboxylic acid methyl ester was used in the following step without any further purification.

1-(2-Methanesulfonyloxy-ethyl)-5,6-dimethoxy-3-(4-methoxy-phenyl)-1H-indole-2-carboxylic acid methyl ester (127 mg, 0.27 mmol) was dissolved in the minimum amount of DMF and 33% methylamine in ethanol (1.5 ml), was added. The solution was left stirring overnight at 60° C. Ethanol was evaporated and the residue was dissolved in diethyl ether (50 ml) and washed with saturated aqueous NH₄Cl (2×20 ml), brine (20 ml), dried over Na₂SO₄ and evaporated under reduced pressure. The crude was purified by flash chromatography (CH₂Cl₂/MeOH, 98:2) to give a yellowish oil, crystallised with pentane/ethyl acetate (100:3) to afford 7,8-dimethoxy-10-(4-methoxyphenyl)-2-methyl-3,4-dihydro-2H-pyrazino[1,2-a]indol-1-one as a light-brown solid (23.7 mg, 24% yield).

ESI POS, spray 3 KV, 20V, 300° C.; MS (m/z): 367 (MH⁺)

¹H-NMR (CDCl₃) δ (ppm): 7.56 (d, 2H), 7.03 (s, 1H), 7.02 (d, 2H), 6.75 (s, 1H), 4.29 (t, 2H), 3.99 (s, 3H), 3.88 (s, 3H), 3.87 (s, 3H), 3.81 (t, 2H), 3.14 (s, 3H). LiAlH₄ (11 mg, 0.29 mmol) was added to a solution of 7,8-dimethoxy-10-(4-methoxyphenyl)-2-methyl-3,4-dihydro-2H-pyrazino[1,2-a]indol-1-one (23.7 mg, 0.06 mmol) in 2 ml of dry THF at 0° C. and the resulting mixture was stirred at room temperature for 3 hours. The reaction was stopped by adding few drops of 1N NaOH. The solution was evaporated under reduced pressure and the residue was purified by flash chromatography on silica gel (CH₂Cl₂/MeOH, 99:1). The collected fractions were concentrated to give a brown solid which was dissolved in diethyl ether and after addition of concentrated HCl, the title compound was isolated in form of hydrochloride, as a white solid (10.7 mg, 43% yield).

ESI POS, spray 3 KV, 20V, 300° C.; MS (m/z): 353 (MH⁺)

¹H-NMR (DMSOd₆) δ (ppm): 11.25 (s, 1H), 7.36 (d, 2H), 7.15 (s, 1H), 7.08 (d, 3H), 4.62 (m, 2H), 4.52 (m, 1H), 4.19 (m, 1H) 3.84 (s, 3H), 3.81 (s, 3H), 3.75 (s, 3H), 3.37 (m, 2H), 2.97 (s, 3H).

### EXAMPLE 66

### 7,8-Dimethoxy-10-(4-methoxyphenyl)-1,2-dihydro-pyrazino[1,2-a]indol-3-one

To a mixture of 5,6-dimethoxy-3-(4-methoxyphenyl)-1H-indol-2-yl-methylamine (98.1 mg, 0.31 mmol), diisopropylethylamine (54 μl, 0.31 mmol) and 5 ml of anhydrous dichloromethane, a solution of trityl chloride (87.5 mg, 0.31 mmol) in 1.5 ml of dry dichloromethane was slowly added at 0° C. The solution was stirred for 2 hours at room temperature. The organic phase washed with water (10 ml) and dried over Na₂SO₄. The solvent was evaporated and the residue was purified on silica gel column cromatography eluting with CH₂Cl₂/Et₃N (0.1%). The collected fractions were evaporated to yield [5,6-dimethoxy-3-(4-methoxy-phenyl)-1H-indol-2-ylmethyl]-trityl-amine as a white solid (132 mg, 74% yield).

ESI POS, spray 3 KV, 20V, 300° C.; MS (m/z): 296.2 (M-TrtNH₂).

To a solution of [5,6-dimethoxy-3-(4-methoxy-phenyl)-1H-indol-2-ylmethyl]-trityl-amine (132 mg, 0.23 mmol) in 3 ml of dry DMF, NaH (12 mg, 0.28 mmol) was added at 0° C. After stirring at room temperature for 1 hour, methyl bromoacetate (26 μl, 0.28 mmol) was added and the solution was stirred for 3 hours. After addition of a saturated solution of NH₄Cl and diethyl ether, the organic layer was separated and washed with brine (20 ml), dried over Na₂SO₄ and evaporated to yield 133 mg of crude {5,6-dimethoxy-3-(4-methoxyphenyl)-2-[(tritylamino)-methyl]-indol-1-yl}acetic acid methyl ester which was used in the following reaction step.

ESI POS, spray 3 KV, 20V, 300° C.; MS (m/z): 627.5 (MH⁺).

{5,6-Dimethoxy-3-(4-methoxy-phenyl)-2-[(tritylamino)-methyl]-indol-1-yl}-acetic acid methyl ester was dissolved in 1.5 ml of a mixture CH₂Cl₂/TFA/TIS (50:50:1). The solution was stirred for 1 hour. The mixture was diluted with dichloromethane (30 ml) and the organic phase was extracted with 1M HCl (2×50 ml). The aqueous phase was treated with 1M NaOH to pH 13 and then extracted with dichloromethane (3×50 ml). The organic layers were dried over Na₂SO₄ and evaporated under reduced pressure to give a residue which was purified by silica gel flash chromatography (CH₂Cl₂/MeOH, 99:1). The title compound was obtained as a light-brown solid (6.3 mg, 11% yield with the respect to [5,6-dimethoxy-3-(4-methoxyphenyl)-1H-indol-2-ylmethyl]-tritylamine).

ESI POS, spray 3 KV, 20V, 300° C.; MS (m/z): 353.3 (MH⁺)

¹H-NMR (CDCl₃) δ (ppm): 7.35 (d, 2H), 7.15 (s, 1H), 7.02 (d, 2H), 6.79 (s, 1H), 4.81 (s b, 2H), 4.75 (s, 2H), 3.97 (s, 3H), 3.90 (s, 3H), 3.87 (s, 3H), 3.80 (d, 1H).

### EXAMPLE 67

### 2-Methanesulfonyl-7,8-dimethoxy-10-(4-methoxyphenyl)-1,2,3,4-tetrahydro-pyrazino[1,2-a]indole

To a solution of 7,8-dimethoxy-10-(4-methoxyphenyl)-1,2,3,4-tetrahydropyrazino[1,2-a]indole (14.5 mg, 0.04 mmol) and N-methyl morpholine (10 μl, 0.05 mmol) in 2 ml of dry dichloromethane a solution of methanesulfonic anhydride (9 mg, 0.05 mmol) in dry dichloromethane (1 ml) was slowly added at 0° C. and the mixture was stirred overnight. The solution was diluted with dichloromethane (30 ml) and washed with 1N HCl (2×5 ml), brine (10 ml), dried over Na₂SO₄ and evaporated under reduced pressure to give the title compound as a pale yellow oil (16.3 mg, 93% yield).

ESI POS, spray 3 KV, 20V, 300° C.; MS (m/z): 417.2 (MH⁺)

¹H-NMR (CDCl₃) δ (ppm): 7.34 (d, 2H), 7.12 (s, 1H), 7.02 (d, 2H), 6.80 (s, 1H), 4.69 (s, 2H), 4.17 (t, 2H), 3.96 (s, 3H), 3.89 (s, 3H), 3.87 (s, 3H), 3.86 (t, 2H), 2.85 (s, 3H).

### EXAMPLE 68

### 7,8-Dimethoxy-10-(4-methoxyphenyl)-2-(propane-2-sulfonyl)-1,2,3,4-tetrahydro-pyrazino[1,2-a]indole

Prepared following process described in Example 67

ESI POS, spray 3 KV, 20V, 300° C.; MS (m/z): 445.3 (MH⁺)

¹H-NMR (CDCl₃) δ (ppm): 7.35 (d, 2H), 7.12 (s, 1H), 7.02 (d, 2H), 6.79 (s, 1H), 4.72 (s, 2H), 4.13 (t, 2H), 3.96 (s, 3H), 3.92 (t, 2H), 3.89 (s, 3H), 3.87 (s, 3H), 3.30-3.18 (m, 1H) 1.34 (d, 6H).

### EXAMPLE 69

### 7,8-Dimethoxy-10-(4-methoxy-phenyl)-2-(toluene-4-sulfonyl)-1,2,3,4-tetrahydro-pyrazino[1,2-a]indole

Prepared following process described in Example 67

ESI POS, spray 3 KV, 20V, 300° C.; MS (m/z): 493.3 (MH⁺)

¹H-NMR (CDCl₃) δ (ppm): 7.69 (d, 2H), 7.31 (d, 4H), 7.08 (s, 1H), 7.03 (d, 2H), 6.71 (s, 1H), 4.47 (s, 2H), 4.08 (t, 2H), 3.93 (s, 3H), 3.88 (s, 3H), 3.87 (s, 3H), 3.64 (t, 2H), 2.41 (s, 3H).

### EXAMPLE 70

### 1-[7,8-Dimethoxy-10-(4-methoxyphenyl)-3,4-dihydro-1H-pyrazino[1,2-a]indol-2-yl]-ethanone

To a solution of 7,8-dimethoxy-10-(4-methoxyphenyl)-1,2,3,4-tetrahydropyrazino[1,2-a]indole (18 mg, 0.05 mmol) and pyridine (10 μl, 0.10 mmol) in 2 ml of anhydrous dichloromethane at 0° C., acetic anhydride (11.2 mg, 0.10 mmol) was added and the reaction mixture was stirred for 6 hours. The mixture was diluted with dichloromethane and washed with 1M HCl, saturated NaHCO₃, brine, dried over Na₂SO₄ and evaporated to give a crude which was purified by silica gel flash chromatography (CH₂Cl₂/MeOH, 98:2). The title compound was obtained as a colourless oil (6.3 mg, 33% yield).

ESI POS, spray 3 KV, 20V, 300° C.; MS (m/z): 381.3 (MH⁺)

¹H-NMR (CDCl₃) δ (ppm): 7.37 (d, 2H), 7.12 (s, 1H), 7.05 (d, 2H), 6.80 (s, 1H), 4.87 (s, 2H), 4.18+4.09 (m, 4H), 3.97 (s, 3H), 3.89 (s, 3H), 3.88 (s, 3H), 2.13 (s, 3H).

### EXAMPLE 71

### 7,8-Dimethoxy-10-(4-methoxyphenyl)-3,4-dihydro-1H-pyrazino[1,2-a]indole-2-carboxylic acid methylamide

7,8-Dimethoxy-10-(4-methoxyphenyl)-1,2,3,4-tetrahydropyrazino[1,2-a]indole (16.4 mg, 0.04 mmol) and N,N-carbonyldiimidazole (9.4 mg, 0.05 mmol) were dissolved in 2 ml of dry THF and refluxed for 1 hour. Solvent was evaporated and the residue dissolved in dichloromethane. The organic phase washed with water and brine. After drying over Na₂SO₄, the solvent was evaporated under reduced pressure and the crude (32 mg) was dissolved in dry acetonitrile. MeI (400 μl, 6.42 mmol) was added and the solution was stirred overnight. After evaporation of the solvent, the residue was dissolved in 3 ml of 2M methylamine in THF and the mixture was stirred for 3 hours. After evaporation of THF, the residue was dissolved in CH₂Cl₂ (50 ml) and washed with 0.5 M HCl, brine, dried over Na₂SO₄, filtered and evaporated to a crude which was purified by silica gel column chromatography, yielding the title compound as a colourless oil (7.3 mg, 38.5% yield).

ESI POS, spray 3 KV, 20V, 300° C.; MS (m/z): 396.2 (MH⁺)

¹H-NMR (CDCl₃) δ (ppm): 7.37 (d, 2H), 7.12 (s, 1H), 7.04 (d, 2H), 6.80 (s, 1H), 4.70 (s, 2H), 4.11 (t, 2H), 4.00 (t, 2H), 3.96 (s, 3H), 3.88 (s, 3H), 3.87 (s, 3H), 2.81 (d, 3H).

### EXAMPLE 72

### 2-Isopropyl-7,8-dimethoxy-10-(4-methoxyphenyl)-1,2,3,4-tetrahydro-pyrazino[1,2-a]indole hydrochloride

To a solution of 7,8-dimethoxy-10-(4-methoxyphenyl)-3,4-dihydro-2H-pyrazino[1,2-a]indol-1-one (73 mg, 0.20 mmol) in 1.5 ml of dry DMF at 0° C., NaH (20 mg, 0.82 mmol) was added and the solution left under stirring for 2 hours. Isopropyl bromide (41 μl, 0.44 mmol) was added and the solution was stirred for 48 hours. The mixture was diluted with diethyl ether (30 ml) and NaH was quenched by adding saturated NH₄Cl. The organic layer washed with brine and dried over Na₂SO₄. Diethyl ether was evaporated to yield 91.0 mg of 2-isopropyl-7,8-dimethoxy-10-(4-methoxyphenyl)-3,4-dihydro-2H-pyrazino[1,2-a]indol-1-one which was used in the following reaction step without any further purification.

ESI POS, spray 3 KV, 20V, 300° C.; MS (m/z): 395.3 (MH⁺).

To a solution of crude 2-isopropyl-7,8-dimethoxy-10-(4-methoxyphenyl)-3,4-dihydro-2H-pyrazino[1,2-a]indol-1-one (91 mg) in 5 ml of dry THF at 0° C., LiAlH₄ (50 mg, 1.52 mmol) was added and the mixture was stirred overnight at r.t. After addition of water and 15% NaOH, the solvent was evaporated and the residue was suspended in dichloromethane. The suspension was filtered and the solution was concentrated to a residue which was purified by silica gel column chromatography (CH₂Cl₂/MeOH, 99:1). The collected fractions were evaporated to give a colourless oil (25 mg). The material was dissolved in diethyl ether and the formation of a precipitate was observed on adding few droplets of 1M HCl in MeOH. The collected solid crystallized upon trituration with diethyl ether/acetone (100:1), to give the title compound (12.5 mg, 16% yield with the respect to lactam).

ESI POS, spray 3 KV, 20V, 300° C.; MS (m/z): 381.3 (MH⁺)

¹H-NMR (CDCl₃) δ (ppm): 13.34 (s, 1H), 7.31 (d, 2H), 7.08 (s, 1H), 7.03 (d, 2H), 6.79 (s, 1H), 4.65 (d, 2H), 4.40-4.28 (m, 2H), 3.95 (s, 3H), 3.92-3.76 (m, 7H), 3.67-3.42 (m, 2H) 1.55-1.43 (d, 6H).

### EXAMPLE 73

### 1-Carbamoylmethyl-5,6-dimethoxy-3-(4-methoxyphenyl)-1H-indole-2-carboxylic acid methyl ester

Fmoc protected Sieber resin (100 mg, 0.017 mmol) was treated twice with 2 ml of a solution of 20% piperidine in DMF (2×5 min) and after washing (DMF), bromoacetic acid (23 mg, 0.17 mmol) and dicyclohexylcarbodiimide (27 ml, 0.17 mmol) in DMF were added. After 2 hours the resin washed with DMF and a solution of NaH (25 mg, 0.625 mmol) and methyl 5,6-dimethoxy-3-(4-methoxyphenyl)-1H-indole-2-carboxylate (73 mg, 0.214 mmol) in 1 ml of dry DMF (previously stirred for 40 min), was added. The product was cleaved from the resin after 24 hours, using a mixture of 7% TFA, 1% TIS, 92% CH₂Cl₂ (3×2 min). After evaporation by nitrogen flow, the crude was purified by silica gel column chromatography (CH₂Cl₂/MeOH 50:1) to afford the title compound (6 mg 88% yield) as a yellowish solid.

ESI POS, spray 3 KV, 20V, 300° C.; MS (m/z): 399.2 (MH⁺)

¹H-NMR (CDCl₃) δ (ppm): 7.43 (d, 2H), 7.05 (d, 1H), 6.77 (s, 1H), 6.71 (s, 1H) 5.35 (s, 2H), 3.97 (s, 3H), 3.89 (s, 3H), 3.82 (s, 3H), 3.79 (t, 2H).

### EXAMPLE 74

### 2-(4,5-Dihydrooxazol-2-yl)-5,6-dimethoxy-3-phenyl-1H-indole

A solution of 5,6-dimethoxy-3-phenyl-1H-indole-2-carboxylic acid (56 mg, 0.188 mmol) in 2 ml of SOCl₂ was stirred at 60° C. for 2 hours. After evaporation of the solvent, the residue was dissolved in dry CH₂Cl₂, and a solution of bromoethylamine (42.8 mg, 0.21 mmol), triethylamine (60 μl, 0.38 mmol) in 2 ml of dry CH₂Cl₂ was added dropwise. The reaction mixture was stirred overnight at r.t. After diluting with CH₂Cl₂, the solution washed with a saturated NaHCO₃, 1M HCl, brine, dried over Na₂SO₄ filtered and concentrated. The resulting oil was purified by silica gel column chromatography (ethyl acetate-CH₂Cl₂ 9:1) to afford the title compound (22 mg, 37% yield).

ESI POS, spray 3 KV, 20V, 300° C.; MS (m/z): 323.1 (MH⁺)

¹H-NMR (CDCl₃) δ (ppm): 9.19 (s, 1H), 7.54 (d, 2H) 7A6 (m, 2H) 7.37 (m, 2H), 6.97 (s, 1H), 6.85 (s, 1H) 4.33 (t, 2H), 4,13 (s, 3H), 3.98-3.94 (m, 10H), 3.85 (s, 3H).

### EXAMPLE 75

### 5,6-Dimethoxy-3-(4-methoxyphenyl)-1-methylcarbamoylmethyl-1H-indole-2-carboxylic acid methyl ester

Sodium hydride (60% mineral oil) (40 mg, 0.98 mmol) was added to a solution of methyl 5,6-dimethoxy-3-(4-methoxyphenyl)-1H-indole-2-carboxylate (280 mg, 0.82 mmol) in DMF (4 ml) at 0° C. and after 1 hour stirring, tert-butyl 2-bromoacetate (133 μl, 0.902 mmol) was added dropwise. The reaction mixture was stirred overnight at r. t. After this time the reaction mixture was diluted with diethyl ether and washed with a saturated solution of NH₄Cl and brine. The organic layer was dried over Na₂SO₄, filtered and evaporated under vacuum. The crude was dissolved in CH₂Cl₂ (20 ml) and a solution of trifluoroacetic acid (5 ml) and Et₃SiH (200 μl) was added. After 3 hours of stirring at r.t. the solvent was evaporated and 310 mg of 2-(2-(methoxycarbonyl)-5,6-dimethoxy-3-(4-methoxyphenyl)-1H-indol-1-yl)acetic acid were obtained.

ESI POS., 3.5 KV, 20V, 300° C.; MS (m/z): 400.3 (MH⁺).

To a solution of 2-(2-(methoxycarbonyl)-5,6-dimethoxy-3-(4-methoxyphenyl)-1H-indol-1-yl)acetic acid (200 mg, 0.501 mmol) in 6 ml of dioxane at 0° C., was added SOCl₂ (141 μL, 2.01 mmol) dropwise and the reaction mixture was stirred at 75° C. for 3 hours. After evaporation of the solvent, the crude was dissolved in dry THF (1 ml) and was added dropwise at 0° C. to a solution of 2M MeNH₂ in THF (10 ml, 20 mmol). The reaction mixture was stirred overnight at r.t. The solvent was evaporated and the crude was purified by silica gel column chromatography (CH₂Cl₂/MeOH, 100:1) to yield the title compound (145 mg, 70%)

ESI POS., 3.5 KV, 20V, 300° C.; MS (m/z): 413.3 (MH⁺)

¹H-NMR (CDCl₃) δ (ppm): 7.36 (d, 2H), 7.00 (d, 2H), 6.88 (s, 1H), 6.87 (s, 1H), 6.23 (d, 1H), 5.05 (s, 2H), 3.98 (s, 3H), 3.89 (s, 3H), 3.83 (s, 3H), 3.69 (s, 3H), 2.00 (d, 3H).

### EXAMPLE 76

### 3,4-Dihydro-7,8-dimethoxy-10-(4-methoxyphenyl)-1H-[1,4]oxazino[4,3-a]indole

To a solution of 2-(2-(methoxycarbonyl)-5,6-dimethoxy-3-(4-methoxyphenyl)-1H-indol-1-yl)acetic acid (100 mg 0.250 mmol) in 3 ml of dry THF under N₂ at 0° C., LiAlH₄ (38 mg, 1.0 mmol) was added and the mixture was stirred at r.t. for 30 min. Afterwards 200 μl of water and 100 μl of 15% NaOH were added, the solvent (THF) was evaporated and the residue was dissolved in CH₂Cl₂. The organic layer washed with brine, dried over NaSO₄, filtered and evaporated under vacuum to afford 85 mg of 2-(2-(hydroxymethyl)-5,6-dimethoxy-3-(4-methoxyphenyl)-1H-indol-1-yl)ethanol.

ESI POS, 3.5 KV, 20V, 300° C.; MS (m/z): 358.2 (MH⁺)

To a solution of 85 mg of 2-(2-(hydroxymethyl)-5,6-dimethoxy-3-(4-methoxyphenyl)-1H-indol-1-yl)ethanol in dry THF (4 ml) at 0° C. NaH (24 mg, 0.59 mmol) was added and the reaction was stirred for 30 min at r.t. Afterwards tosylimidazole (26 mg, 0.119 mmol) was added at 0° C. and the reaction mixture was stirred for additional 30 min. After this time a saturated solution of NH₄Cl was added to the reaction mixture and THF was evaporated. The crude was dissolved in CH₂Cl₂ and washed with brine. The aqueous phase was extracted several times with CH₂Cl₂ and the organic layers were dried over NaSO₄ filtered and evaporated to afford a crude that was chromatographed over silica gel (Petroleum ether/ethyl acetate, 8:2-1:1). to afford 42 mg (49% yield) of the title compound.

ESI POS 3.5 KV, 20V, 300° C.; MS (m/z): 340.3 (MH⁺)

¹H-NMR (CDCl₃) δ (ppm): 7.34 (d, 2H), 7.18 (s, 1H), 7.01 (d, 2H), 6.82 (s, 1H), 5.02 (s, 2H), 4.21 (t, 2H), 4.07 (t, 2H), 3.97 (s, 3H), 3.90 (s, 3H), 3.87 (s, 3H).

### EXAMPLE 77

### 5,6-Dimethoxy-3-(4-methoxyphenyl)-2-(5-methyl-1,3,4-oxadiazol-2-yl)-1H-indole

350 mg (1.026 mmol) of 5,6-dimethoxy-3-(4-methoxyphenyl)-1H-indole-2-carbohydrazide (prepared as described in Example 26) was dissolved in 5 ml of acetic acid and refluxed for 3 hours. The solution was diluted with dichloromethane and washed with water. The organic layer was dried over NaSO₄, filtrated and evaporated to afford crude N′-acetyl-5,6-dimethoxy-3-(4-methoxyphenyl)-1H-indole-2-carbohydrazide (165 mg) that was used in the next step without any further purification.

ESI POS, 3.5 KV, 20V, 300° C.; MS (m/z): 384.2 (MH⁺)

A solution of N′-acetyl-5,6-dimethoxy-3-(4-methoxyphenyl)-1H-indole-2-carbohydrazide (160 mg, 0.41 mmol) in POCl₃ (700 μl) was stirred at 80° C. for 35 min. After evaporation of the solvent the residue was dissolved in dichloromethane and washed with water. The organic layer was dried over NaSO₄ filtered and evaporated. The crude was chromatographed (Petroleum ether/ethyl acetate, 8:2) to afford 27.5 mg (19% yield) of the title compound.

ESI POS, 3.5 KV, 20V, 300° C.; MS (m/z): 366.2 (MH⁺)

¹H-NMR (CDCl₃) δ (ppm): 9.52 (s, 1H), 7.04 (d, 2H), 7.48 (d, 2H), 6.97 (s, 1H), 6.95 (s, 1H), 3.97 (s, 3H), 3.91 (s, 3H), 3.87 (s, 3H), 2.47 (s, 3H).

### EXAMPLE 78

### 5-Hydroxy-3-phenyl-1H-indole-2-carboxylic acid ethyl ester

A solution of 5-methoxy-3-phenyl-1H-indole-2-carboxylic acid ethyl ester (295 mg, 1 mmol) in 5 ml of CH₂Cl₂ was added dropwise at room temperature and under a nitrogen atmosphere to a solution of BBr₃ (260 μl, 2.75 mmol) in 20 ml of CH₂Cl₂. The reaction mixture was stirred 1 h, then it was cooled to −5° C. and 4 ml of EtOH were added. The solvents were evaporated and the resulting residue was crystallized from (i-Pr)₂O, yielding 208 mg (74% yield) of the title compound.

ESI POS, 3.2 KV, 20V, 300° C.; MS (m/z): 282.1 (MH⁺)

¹H-NMR (CDCl₃) δ (ppm): 8.85 (s br, 1H); 7.52 (dd, 2H); 7.43 (dd, 2H); 7.38 (dd, 1H); 7.31 (d, 1H); 6.98 (d, 1H); 6.96 (dd, 1H); 4.56 (s br, 1H); 4.27 (q, 2H); 1.22 (t, 3H).

### EXAMPLE 79

### 3-Pyridin-3-yl-1H-indole-2-carboxylic acid ethyl ester

A solution of 0.2 g (0.63 mmol) of 3-iodoindole-2-carboxylic acid ethyl ester (prepared as described in Chem. Pharm. Bull. 36(6), 2248-2252, 1988) in 1,2-dimethoxyethane (5 ml) was treated with [1,1′-bis-(diphenylphospino) ferrocenedichloropalladium(II)] (25.3 mg, 0.031 mmol) and the resulting mixture was degassed under vacuum for 5 min. Pyridine-3-boronic acid (0.085 g, 0.69 mmol) was added followed by 2M K₂CO₃ aqueous solution (0.78 ml, 1.57 mmol). The reaction mixture was stirred at 85° C. under N₂ for 18 h. The solvent was evaporated under reduced pressure. The residue was dissolved in DCM (2 ml) and directly loaded on silica cartridge (5 g). Elution with petroleum ether/ethyl acetate (10:0 to 1:1) afforded the title compound as a pale yellow solid (0.124 g, 74% yield).

ESI POS, 3.2 KV, 20V, 300° C.; MS (m/z): 267.1 (MH⁺)

¹H-NMR (CDCl₃) δ (ppm): 9.27 (s br, 1H); 8.81 (d, 1H); 8.63 (dd, 1H); 7.91 (ddd, 1H); 7.60 (d, 1H); 7.51-7.35 (m, 3H); 7.18 (ddd, 1H); 4.31 (q, 2H); 1.24 (t, 3H).

### EXAMPLE 80

### 3-Phenyl-1H-indole-2-carboxylic acid ethyl ester

Prepared as described in Example 79

ESI POS, 3.2 KV, 20V, 300° C.; MS (m/z): 266.2 (MH⁺)

¹H-NMR (CDCl₃) δ (ppm): 9.00 (s br, 1H); 7.64 (dd, 1H); 7.56 (dd, 2H); 7.49-7.33 (m, 5H); 7.15 (ddd, 1H); 4.30 (q, 2H); 1.24 (t, 3H).

### EXAMPLE 81

### 5,6-Dimethoxy-3-pyridin-4-yl-1H-indole-2-carboxylic acid ethyl ester

Prepared following process described in EP 449196

ESI POS, 3.2 KV, 20V, 300° C.; MS (m/z): 327.2 (MH⁺)

¹H-NMR (CDCl₃) δ (ppm): 9.11 (s br, 1H); 8.70 (s br, 2H), 7.52 (d, 2H); 6.90 (d, 2H); 4.29 (q, 2H); 3.95 (s, 3H); 3.86 (s, 3H); 1.25 (t, 3H).

Compounds described in Examples 1-81 are summarised in Table 1

TABLE 1

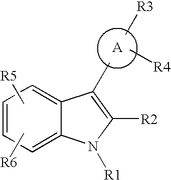

| Example | R₁ | R₂ | A | R₃ | R₄ | R₅ | R₆ |
|---|---|---|---|---|---|---|---|
| 1 | H | COOMe | Ph | p-OMe | H | 5-OMe | 6-OMe |
| 2 | H | COOMe | Ph | p-OMe | m-OMe | 5-OMe | 6-OMe |
| 3 | H | COOMe | Ph | H | H | 5-OMe | 6-OMe |
| 4 | H | COOEt | Ph | H | H | 5-OMe | 6-OMe |
| 5 | H | COOMe | Ph | p-OMe | H | H | H |
| 6 | CH₂Ph | COOMe | Ph | p-OMe | H | 5-OMe | 6-OMe |
| 7 | CH₂COOCH₃ | COOMe | Ph | p-OMe | H | 5-OMe | 6-OMe |
| 8 | CH₂CON(CH₃)₂ | COOMe | Ph | p-OMe | H | 5-OMe | 6-OMe |
| 9 | CH₂CH₂CH₃ | COOMe | Ph | p-OMe | H | 5-OMe | 6-OMe |
| 10 | CH₂CN | COOMe | Ph | p-OMe | H | 5-OMe | 6-OMe |
| 11 | CH₂CH₂N(CH₃)₂ | COOMe | Ph | p-OMe | H | 5-OMe | 6-OMe |
| 12 | CH₂CH₂OH | COOMe | Ph | p-OMe | H | 5-OMe | 6-OMe |
| 13 | H | COOH | Ph | p-OMe | H | 5-OMe | 6-OMe |
| 14 | H | CONH₂ | Ph | p-OMe | H | 5-OMe | 6-OMe |
| 15 | H | CH₂NH₂ | Ph | p-OMe | H | 5-OMe | 6-OMe |
| 16 | H | CH₂NHCOCH₃ | Ph | p-OMe | H | 5-OMe | 6-OMe |
| 17 | H | CN | Ph | p-OMe | H | 5-OMe | 6-OMe |
| 18 | CH₂CH₂N(CH₃)₂ | CN | Ph | p-OMe | H | 5-OMe | 6-OMe |
| 19 | H | CONHSO₂CH₃ | Ph | p-OMe | H | 5-OMe | 6-OMe |
| 20 | H | CH₂OH | Ph | p-OMe | H | 5-OMe | 6-OMe |
| 21 | H | CH₂NHCH₃ | Ph | p-OMe | H | 5-OMe | 6-OMe |
| 22 | H | CH₂CH₂COOEt | Ph | p-OMe | H | 5-OMe | 6-OMe |
| 23 | H | 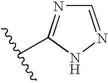 | Ph | p-OMe | H | 5-OMe | 6-OMe |
| 24 | H | 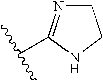 | Ph | p-OMe | H | 5-OMe | 6-OMe |
| 25 | H | 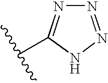 | Ph | p-OMe | H | 5-OMe | 6-OMe |
| 26 | H | 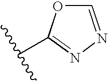 | Ph | p-OMe | H | 5-OMe | 6-OMe |
| 27 | —CH₂CH₂NHCO— | | Ph | p-OMe | H | 5-OMe | 6-OMe |
| 28 | —CH₂CH₂NHCH₂— | | Ph | p-OMe | H | 5-OMe | 6-OMe |
| 29 | H | COOH | Ph | H | H | 5-OMe | 6-OMe |
| 30 | H | COOMe | Ph | p-Cl | H | 5-OMe | 6-OMe |
| 31 | H | COOMe | Ph | p-CF3 | H | 5-OMe | 6-OMe |
| 32 | H | COOMe | Ph | p-CH3 | H | 5-OMe | 6-OMe |
| 33 | H | COOMe | Ph | p-F | H | 5-OMe | 6-OMe |
| 34 | H | COOMe | Ph | o-Cl | H | 5-OMe | 6-OMe |
| 35 | H | COOMe | Ph | m-Cl | H | 5-OMe | 6-OMe |
| 36 | H | COOEt | Ph | H | H | 5-Cl | H |
| 37 | H | COOEt | Ph | H | H | 5-F | H |
| 38 | H | COOEt | Ph | H | H | 5-OMe | H |
| 39 | CH₂CH₂OMe | COOMe | Ph | p-OMe | H | 5-OMe | 6-OMe |
| 40 | CH₂CH₂OH | CN | Ph | H | H | 5-OMe | 6-OMe |
| 41 | CH₂CH₂CH₂OH | COOMe | Ph | p-OMe | H | 5-OMe | 6-OMe |
| 42 | CH₂CH₂OH | CN | Ph | p-OMe | H | 5-OMe | 6-OMe |
| 43 | CH₂CH₂OH | 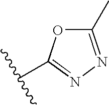 | Ph | p-OMe | H | 5-OMe | 6-OMe |
| 44 | CH₂CH₂CH₂OH | CN | Ph | p-OMe | H | 5-OMe | 6-OMe |
| 45 | H | CONH2 | Ph | H | H | 5-OMe | 6-OMe |
| 46 | H | CONMe₂ | Ph | H | H | 5-OMe | 6-OMe |
| 47 | H | CON(CH₂CH₂)₂O | Ph | H | H | 5-OMe | 6-OMe |
| 48 | H | CONHMe | Ph | H | H | 5-OMe | 6-OMe |
| 49 | H | CN | Ph | H | H | 5-OMe | 6-OMe |
| 50 | CH₂CH₂CH₃ | CN | Ph | H | H | 5-OMe | 6-OMe |
| 51 | CH₂CH₂N(CH₃)₂ | CN | Ph | H | H | 5-OMe | 6-OMe |
| 52 | CH₂CH₂N(CH₂)₄ | COOMe | Ph | p-OMe | H | 5-OMe | 6-OMe |
| 53 | CH₂CH₂CH₂N(CH₃)₂ | COOMe | Ph | p-OMe | H | 5-OMe | 6-OMe |
| 54 | CH₂CH₂N(CH₂)₄ | CN | Ph | p-OMe | H | 5-OMe | 6-OMe |
| 55 | CH₂CH₂CH₂N(CH₃)₂ | CN | Ph | p-OMe | H | 5-OMe | 6-OMe |
| 56 | CH₂CH₂N(CH₂CH₂)₂O | CN | Ph | p-OMe | H | 5-OMe | 6-OMe |
| 57 | CH₂CH₂N(CH₃)₂ | 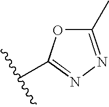 | Ph | p-OMe | H | 5-OMe | 6-OMe |
| 58 | H | 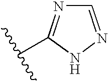 | Ph | H | H | 5-OMe | 6-OMe |
| 59 | —CH₂CH₂NHCO— | | Ph | H | H | 5-OMe | 6-OMe |
| 60 | CH₂CH₂NH₂ | COOMe | Ph | p-OMe | H | 5-OMe | 6-OMe |
| 61 | —CH₂CH₂NHCH₂— | | Ph | H | H | 5-OMe | 6-OMe |
| 62 | CH₂CH₂N(CH₂CH₂)₂NCH₃ | COOMe | Ph | p-OMe | H | 5-OMe | 6-OMe |
| 63 | CH₂CH₂N(CH₂CH₂)₂O | COOMe | Ph | p-OMe | H | 5-OMe | 6-OMe |
| 64 | CH₂CH₂N(CH₂CH₂)₂NCH₃ | CN | Ph | p-OMe | H | 5-OMe | 6-OMe |
| 65 | —CH₂CH₂N(CH₃)CH₂— | | Ph | p-OMe | H | 5-OMe | 6-OMe |
| 66 | —CH₂CONHCH₂— | | Ph | p-OMe | H | 5-OMe | 6-OMe |
| 67 | —CH₂CH₂N(SO₂CH₃)CH₂— | | Ph | p-OMe | H | 5-OMe | 6-OMe |
| 68 | —CH₂CH₂N(SO₂i-Pr)CH₂— | | Ph | p-OMe | H | 5-OMe | 6-OMe |
| 69 | —CH₂CH₂N(SO₂C₆H₄p-CH₃)CH₂— | | Ph | p-OMe | H | 5-OMe | 6-OMe |
| 70 | —CH₂CH₂N(COCH₃)CH₂— | | Ph | p-OMe | H | 5-OMe | 6-OMe |
| 71 | —CH₂CH₂N(CONHCH₃)CH₂— | | Ph | p-OMe | H | 5-OMe | 6-OMe |
| 72 | —CH₂CH₂N(i-Pr)CH₂— | | Ph | p-OMe | H | 5-OMe | 6-OMe |
| 73 | CH₂CONH₂ | COOMe | Ph | p-OMe | H | 5-OMe | 6-OMe |
| 74 | H | 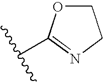 | Ph | H | H | 5-OMe | 6-OMe |
| 75 | CH₂CONHCH₃ | COOMe | Ph | p-OMe | H | 5-OMe | 6-OMe |
| 76 | —CH₂CH₂OCH₂— | | Ph | p-OMe | H | 5-OMe | 6-OMe |
| 77 | H | 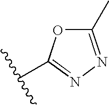 | Ph | p-OMe | H | 5-OMe | 6-OMe |
| 78 | H | COOEt | Ph | H | H | 5-OH | H |
| 79 | H | COOEt | 3-pyridyl | H | H | H | H |
| 80 | H | COOEt | Ph | H | H | H | H |
| 81 | H | COOEt | 4-pyridyl | H | H | 5-OMe | 6-OMe |

### In Vitro Studies

1. Biochemistry

1.1 Determination of Vacuolar ATpase Inhibition in Human Osteoclastoma (hOc)

Osteoclast-like giant cells isolated from human osteoclastoma are homogenized using a glass-teflon homogeniser (1000 rpm) and the material is centrifuged for 20 minutes at 6000 g. The resultant pellet is resuspended and centrifuged at 100000 g for 60 minutes to sediment the microsomal fraction. The resultant pellet is resuspended in medium at pH 7.4 and stored under liquid nitrogen.

Inhibition of bafilomycin sensitive ATPase activity is assayed by measuring the release of inorganic phosphate during 30 minutes of incubation, at 37° C., of the human osteoclastoma microsomal fraction in 96-well plates. The reaction medium contains 1 mM ATP, 10 mM Hepes-Tris buffer pH 8, 50 mM KCl, 5 μM valinomycin, 5 μM nigericin, 1 mM CDTA-Tris, 100 μM ammonium molybdate, 0.2 M sucrose and the microsomal fraction (20 μg protein/ml). The reaction is initiated by adding MgSO₄ and terminated, after 30 minutes, by adding 4 volumes of the reagent malachite green, prepared according to Chan K., Anal. Biochem. 157, 375-380, 1986.

1.2 Determination of Vacuolar ATpase Inhibition in the Bovine Chromaffin Cells Membranes (BCG)

About 20 adrenal glands (Cidon S., J. Biol. Chem. 258, 2892-2898, 1983) are removed from healthy bovines; the medulla is quickly separated from the cortex, which is discarded. The medulla is homogenised at 4° C. with a suitable medium at pH 7.5, then filtered. The remaining solid material is further homogenised, filtered and recombined with the preceding filtrate, resuspended and centrifuged at 1000 g for 15 minutes; the supernatant obtained is centrifuged at 10000 g for 20 minutes. The resultant pellet is resuspended and stratified through a sucrose gradient formed of a lower part of 15 ml 1.5 M sucrose and an upper part of 10 ml 1.2 M sucrose. After overnight centrifugation at 4° C. with a SW28 rotor at 20000 rpm, the chromaffiln cells sediment into a pellet. This latter is resuspended, centrifuged at 3000 g for 10 minutes, and the supernatant obtained is centrifuged at 200000 g for 60 minutes. The pellet is then resuspended in 4 ml of a suitable medium containing 0.2 μg/ml pepstatin A and 0.4 μg/ml leupeptin and stored under liquid nitrogen.

The method for ATPase inhibition assay is the same as that followed for the osteoclastoma.

2. Cell Pharmacology

2.1 Cell Lines and Culture Conditions

Human Colon Carcinoma:

HT29 and HT29/Mit (line obtained by prolonged exposure to mitoxantrone, and characterised by overexpression of BCRP, which confers cross resistance to topotecan, irinotecan and to its metabolite SN38): maintained in McCoy 5A medium+10% FCS.

LoVo and LoVo/Dx (line obtained by prolonged exposure to doxorubicin and characterised by overexpression of P-glycoprotein, which confers resistance to doxorubicin): maintained in HAM-F12 medium+10% FCS. HCT116 maintained in RPMI 1640 medium+10% FCS.

Human Neuroblastoma:

SH-SY5Y and SK-N-BE(2): maintained in HAM-F12 medium+10% FCS.

Human Hepatic Carcinoma:

HepG2: maintained in EMEM medium+10% FCS.

Human Ovarian Carcinoma:

A2780: maintained in RPMI 1640 medium+10% FCS.

Human Lung Carcinoma:

H460: maintained in RPMI1640 medium+10% FCS.

2.2 Scheme of the Antiproliferative Activity Experiment (Treatment Time: 72 Hours)

The cells (HT29 and HT29/Mit: 40,000 cells/ml, LoVo, LoVo/Dx and HCT116: 50,000 cells/ml) are seeded in 100 μl of the respective culture media in 96-well plates. 24 hours after seeding, an aliquot (10 μl) of drug at the various concentrations is added. In the samples in which the effect of the combination of two compounds is to be tested the inhibitor is added immediately before the cytotoxic. For each dose or combination of doses/drugs the effect of the treatment is determined in 4-8 replicates.

After 72 hours of treatment the antiproliferative effect is evaluated using the sulforhodamine B (SRB) assay: the cells are fixed by adding 25 μl of 50% TCA to each well and left for 1 hour at 4° C. After washing them with water and allowing them to dry, 100 μl of 0.4% SRB in 1% acetic acid are added and left for 30 minutes at room temperature. After 4 washes in 1% acetic acid, they are left to dry then the dye fixed by the proteins is dissolved under basic conditions with 100 μl 10 mM cold Tris and the solution is read using a spectrophotometer at 550 nm.

Data Analysis

Percentage cell growth is calculated as the optical density of treated samples compared to the optical density of controls (untreated cells).

The Combination Index (C.I.) was determined according to Kern's method, 1988 (who continued from Drewinko B, 1976) (Kem D. H., Cancer Res. 48, 117-121, 1988; Drewinko B., Cancer Biochem. Biophys. 1, 187-195, 1976) by means of the following formula: (Sfa+SFb)/Sfab, where SFa and SFb are the fractions of cells surviving to treatment with compound a and b, respectively; SFab is the fraction surviving to the combination of compounds a and b. If the result is =1, the interaction between the two compounds is additive; if it is >1, the interaction between the two compounds is synergistic; if it is <1, the interaction between the two compounds is antagonistic.

2.3 Scheme of the Antiproliferative Activity Experiment (Treatment Time: 48 Hours)

The cells (concentration: 30,000 cells/ml) are seeded in 90 μl of the respective culture media in 96-well plates. 24 hours after seeding, an aliquot (10 μl) of the drug at the various concentrations is added (for each concentration there are 3 replicates). After 48 hours of treatment the antiproliferative effect is evaluated with a luminescence assay (Perkin Elmer Life Sciences ATPlite):

50 μl of a lysis solution are added to each well followed by an equal volume of a solution containing luciferase and D-luciferin. The ATP present in all the metabolically active cells brings about the transformation reaction of D-luciferin, catalysed by luciferase, to produce a luminescent signal as described in the following scheme:
ATP+D-luciferin+O₂→Oxyluciferin+AMP+PP₁+CO₂+Light

The luminescence produced (expressed in counts per second, CPS) is measured by means of a microplate scintillation analyzer (Perkin Elmer Life Sciences Top Count).

Data Analysis

Percentage inhibition of luminescence in the treated cells compared to the control is calculated; concentration-response curves are then analysed using Grafit v.5.0.

2.4 Scheme of Apoptosis Experiment

1.5×10⁶ cells/sample are seeded. After 24 hours the cells are treated with the compounds for 48 or 72 hours. At the end of the treatment, the cells are detached with Trypsin/EDTA, washed in PBS (phosphate buffered solution) and incubated for 45 minutes at room temperature in 1 ml of 4% paraformaldehyde. The cells are then washed with PBS and resuspended in 100 μl of permeabilizing solution (0.1% triton in 0.1% sodium citrate) for 2 minutes in ice. After a further wash, the cells are resuspended in 50 μl of Tunel reaction mix (Boehringer Mannheim) and left at 37° C. for 1 hour in the dark. After washing in PBS, the cells are resuspended in PBS and analysed by cytofluorimeter or examined by fluorescence microscope.

2.5 Scheme of Irradiation Experiment

HT29 cells (50,000 cells/ml) were seeded and 24 h later they were irradiated with a 137Cs source delivering 0.13 Gy/s, in presence and in absence with the test compound. After 72 h treatment, adherent cells were collected, washed in PBS and counted to evaluate the cytotoxic effect of the treatment.

2.6 Scheme of migration and invasion assays

H460 cells were seeded in complete medium and treated with different compound concentrations for 24 h. Then, cells were harvested and transferred to 24-well Transwell chambers (Costar) in serum-free medium in the following ways:
migration assay: 1.2×10⁵ cells/well were seeded in the upper chamber, and the drug was added, in the same concentrations utilized before, in both upper and lower chambers. After 4 h of incubation at 37° C., migrated cells were fixed in 95% ethanol, stained with a 2% crystal violet in 70% ethanol solution, and counted by an inverted microscope.
invasion assay: Transwell membranes were coated with 12.5 μg/well of Matrigel (BD Biosciences) and dried for 24 h. After this, 2.4×10⁵ cells/well were seeded onto the artificial basement membrane in upper chamber, and drug was added as described for migration assay. After 24 h of incubation at 37° C., cells that invaded the Matrigel and migrated to the lower chamber were stained and counted as described for migration assay.
   2.7 Results
   Antiproliferative Effect on Tumour Cells (Single Treatment)

The results are given in the following Table 2

| | | ATPase | | Antiproliferative activity | | |
|---|---|---|---|---|---|---|
| | | activity | | (IC₅₀ ± S.D., μM) | | |
| | | (IC₅₀, μm) | | HT-29 | | HepG2 |
| Compound | Structure | hOc | BCG | 72 h treatment | HT-29/Mit | 48 h treatment |
| Cisplatin | | | | 6.8 | | >25 |
| Topotecan | | | | 0.07 ± 0.01 | 50.6 | |
| Bafilomycin A1 | | 0.0001 | | 0.003 ± 0.002 | 0.005 | <0.006 |
| Example 1 | 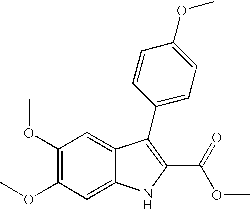 | 0.500 | 0.644 | 14.4 ± 2.1 | 1.0 ± 0.9 | 24.4 ± 7.9 |
| Example 2 | 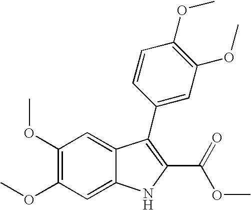 | | | 35.2 ± 5.2 | 15.2 ± 0.3 | 25.5 ± 4.5 |
| Example 3 | 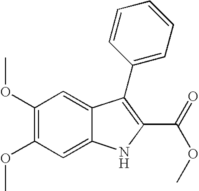 | | 0.628 | 37.3 | 0.15 | |
| Example 4 | 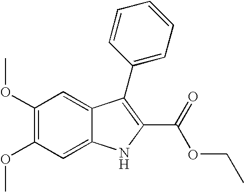 | | 0.350 | 37.3 | <0.1 | |
| Example 12 | 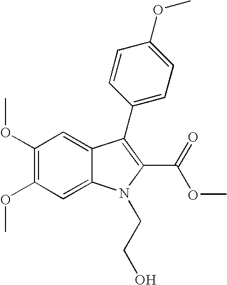 | | 0.247 | 4.6 | 0.66 ± 0.30 | |
| Example 17 | 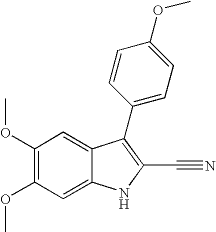 | | 0.273 | 4.9 ± 0.5 | 2.5 | |
| Example 18 | 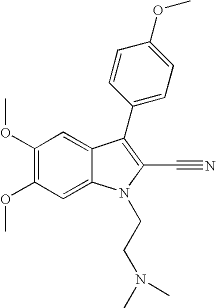 | | 0.567 | 16.7 | 0.15 | |
| Example 23 | 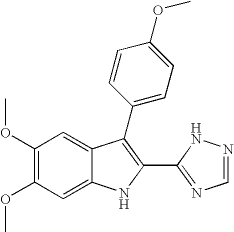 | | 0.470 | 41.9 | 2.3 | |
| Example 26 | 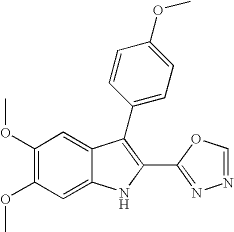 | | 0.710 | 40.3 | 2.7 | |
| Example 27 | 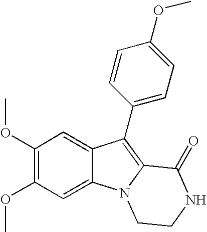 | | 0.248 | 5.2 | 2.9 | |
| Example 28 | 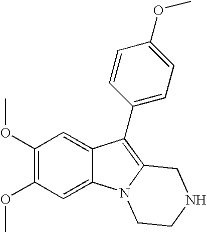 | | 1.269 | 37.3 | 0.53 | |

The cell pharmacology study was conducted in the system comprising the original HT29 line and its variant HT29/Mit, selected in the presence of mitoxantrone and characterised by overexpression of the BCRP transport system. This phenotype confers cross-resistance to topotecan and irinotecan (and its metabolite SN38). The parental line produces tumours in nude mice which are poorly sensitive to topotecan and to DNA topoisomerase inhibitors. The state of resistance is further increased in the model selected for resistance to mitoxantrone (HT29/Mit).

The comparative study of the two cell lines has highlighted a surprising activity (after 72 hours of treatment) of the compounds of examples 1, 3, 4, 12, 18, 23, 26, 28 in the resistant line with IC₅₀ values within the range 0.1-3 μM being substantially lower than those found in the sensitive line, with IC₅₀ values in the range 5-42 μM. The fact that the antiproliferative activity of the aforecited compounds increases in a very significant manner (from 7 to 370 times) in the resistant line compared to the sensitive line strongly support their therapeutic use in resistant tumours, also administered alone.

Antiproliferative Effect on Tumour Cells (Combined Treatment with Known Antitumour Agents)

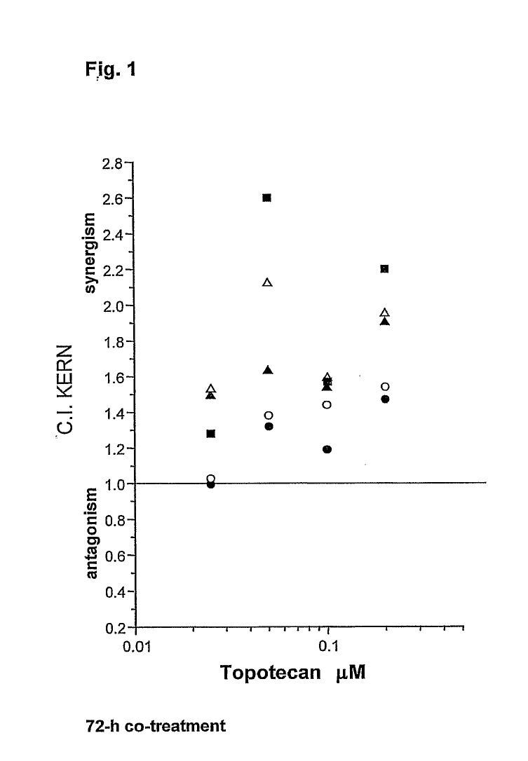

The compound of example 1 has produced marked synergistic effects in combination with numerous cytotoxic agents of interest in clinical therapy, such as topotecan, SN38, taxol, doxorubicin and anthracyclines. The synergism is particularly evident at subtoxic doses of the inhibitor (4-8 μM) on the activity of topotecan in the tumour cell line HT29, as illustrated in FIG. 1. The combination of topotecan with the compound of example 1 at two concentrations (4 μM—3 experiments; 8 μM—2 experiments) highlight a clear synergistic effect, the data being expressed as Combination Index according to Kern (see in vitro studies 2.2).

In addition, the compound of example 1 has shown to be particularly effective in enhancing the activity of topotecan in the resistant model (HT29/Mit), as it causes synergism at non-toxic concentrations in a wide range of pharmacologically significant concentrations (0.01-0.1 μM).

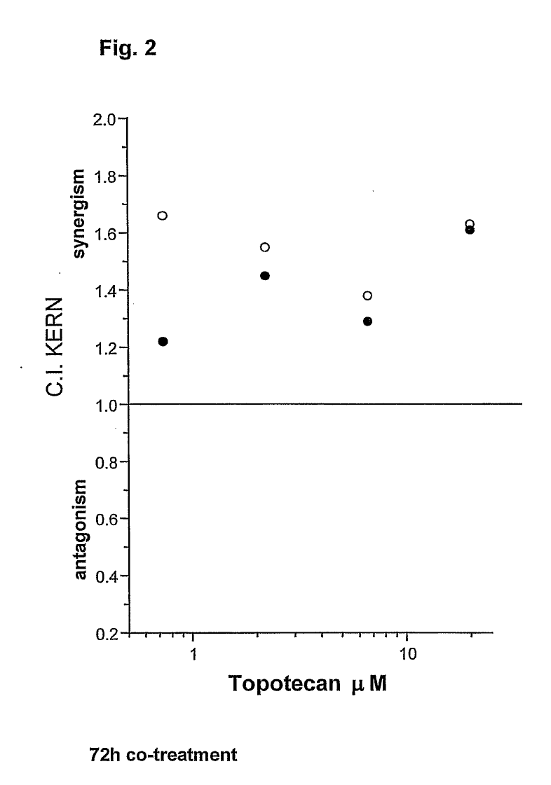

In FIG. 2, it can be seen that the co-treatment of HT29/Mit cells with the compound of example 1 (at two concentrations which are not active by themselves) and with topotecan produces a clear synergistic effect.

It is extremely interesting that the compounds of examples 12, 23, and 26 demonstrated synergistic effects in combination with topotecan both in HT29 and HT29/Mit cells.

In Vivo Studies

1.1 Model of HT29/Mit human colon carcinoma xenografts-Antitumor activity

Female athymic Swiss nude mice (8-10 weeks old) (Charles River, Calco, Italy) were used for the experiments.

The animals were maintained at constant temperature and humidity, and were allowed to eat and drink freely. The experimental protocol was approved by the Ethics Committee for Animal Experimentation of the Istituto Nazionale Tumori of Milan.

The antitumour effectiveness of the compounds of the invention under discussion was tested on athymic mouse models implanted with HT29 and/or HT29/Mit tumour cells: this latter variety is highly resistant to topotecan treatment. The tumour cells were implanted in vivo via subcutaneous injection of 10⁷ cells taken from in vitro cultures. Randomized groups of five mice with bilateral subcutaneous tumours were used for the experiment.

Topotecan or other known antitumour agents (dissolved in distilled water or an appropriate solvent) and the compounds of the invention (dissolved in Cremophor EL: ethanol: saline solution in the proportions 5:5:90, or in an appropriate solvent) were administered orally from the third day, alone or in combination, in agreement with a treatment scheme selected in an appropriate manner depending on the type of compound to be studied.

The weight (or volume) of the tumour in treated mice compared to controls is represented graphically on the y-axis against time (x-axis).

1.2 Model of H460 Human Non-Small Cell Lung Carcinoma Xenograft-Antitumor and Antimetastatic Activities

Female athymic Swiss nude mice (8-10 weeks old) (Charles River, Calco, Italy) were used for the experiments, as described above.

H460 cells were injected i.p. into nude mice, adapted to grow as ascitis and maintained in vivo by i.p. passages (5×10⁶ cells/mouse in 0.5 ml PBS) (Pratesi G., Br. J. Cancer 63, 71-74, 1991). Briefly, cells were collected from the donor mice about 7 days after inoculum. After washing, cell number and viability were determined by trypan blue exclusion. Such process allowed to obtain a single cell suspension easily available for s.c or i.v. injection.

The effects of the compounds of the invention and/or topotecan on the growth of primary tumors and spontaneous lung metastasis were tested in mice inoculated s.c. in the right flank with H460 ascitic tumor cells (2×10⁶/mouse). Each control or drug-treated group included 9-11 mice. The s.c. tumor growth was followed by biweekly measurements of tumor diameters with a Vernier caliper. Drug treatment was delivered orally, for 8 weeks, from day 1. Topotecan was delivered at the dose of 1 mg/kg and compounds of the invention were delivered at the dose of 30 mg/kg; in the combination group the compounds of the invention were delivered almost 1 hour after topotecan treatment. Control mice were solvent-treated orally in parallel with drug treatments.

Drug efficacy was assessed as mean percentage tumor weight inhibition in drug-treated versus control mice expressed as tumor weight inhibition % (TWI %)=100−(mean tumor weight treated/mean tumor weight control×100), evaluated during and after drug treatment. Drug tolerability was assessed in tumor-bearing mice as either lethal toxicity, i.e., any death in treated mice occurring before any control death, or percentage body weight loss (BWL %)=100−(body weight on day x/body weight on day 1×100), where x represents a day after or during the treatment period.

At day 63, tumor-bearing mice were sacrificed by cervical dislocation and their lungs were removed and weighed. Lung lobes were spliced between two glass slides and the metastatic nodules were macroscopically counted against a bright light (Corti C, J. Cancer Res. Clin. Oncol. 122, 154-60, 1996). Spontaneous lung metastases were present in 100% of control mice. Reading of metastasis was performed by two independent observers, unaware of the experimental group, with an interobserver reproducibility >95%. The metastatic nature of these areas was confirmed by histological analysis of digital images obtained by Image Analysis System software (Delta System, Rome, Italy).

1.3 Results

Antitumor Activity (HT291Mit Xenograft Model)

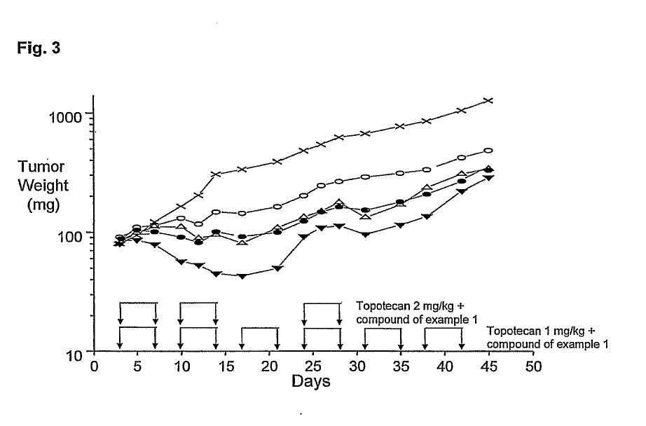

In vivo results have been displayed in FIG. 3.

In HT29/Mit xenograft in vivo model, the oral treatment with topotecan (1 and 2 mg/kg, schedule described in FIG. 3) produced a clear antitumor effect (62% and 80% tumor growth inhibition) at 1 and 2 mg/kg respectively. The combined administration with the compound of example 1 given at 30 mg/kg p.o. with topotecan produced an increased antitumoral efficacy (statistically significant when 1 mg topotecan was utilized).

Compound of example 1, administered alone in the same model (data not shown), was able to produce a 43% statistically significant tumor growth inhibition at 30 mg/kg p.o. after 7 days of treatment.

1. Method for treating or preventing tumours comprising administering to a patient in need thereof, a compound of formula (I) 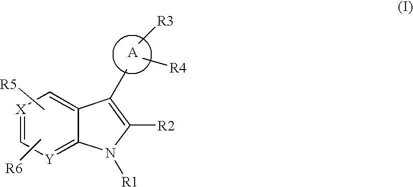
wherein:
R1 is chosen from H, alkyl, arylalkyl, hydroxyalkyl, alkoxyalkyl, aminoalkyl, alkylaminoalkyl, dialkylaminoalkyl, alkoxycarbonylalkyl, aminocarbonylalkyl, alkylaminocarbonylalkyl, dialkylaminocarbonylalkyl, cyanoalkyl, or a group R′R″Nalkyl, in which R′ and R″, together with the nitrogen atom to which they are attached, may form a 5, 6 or 7 membered ring, optionally containing a heteroatom chosen from O, S and N, and where said N atom may be substituted by alkyl;
R2 is chosen from heterocyclyl optionally substituted by alkyl or aryl, ester, amide, nitrile; or R1 and R2 together form a 5, 6 or 7 membered ring containing optionally a heteroatom chosen from O, S, N and containing optionally a carbonyl function which can be attached to any carbon atom of said ring, and where said N atom may be substituted by alkyl, aryl, arylalkyl, heteroaryl, alkylsulfonyl, arylsulfonyl, alkylcarbonyl, arylcarbonyl, alkylaminocarbonyl, arylaminocarbonyl;
R3, R4, R5, R6 each independently represent H, alkyl, alkoxy, hydroxy, halogen, trifluoromethyl, trifluoromethyloxy;
X and Y each independently represent carbon;
A is phenyl,
where in all substituents referred above, the term “aryl” means phenyl,
in the preparation of a medicament useful for the treatment of tumours.

2. Method as claimed in claim 1, wherein R1 is selected from Me, Et or Pr, benzyl, hydroxyethyl, hydroxypropyl, methoxyethyl, aminoethyl, methylaminoethyl, dimethylaminoethyl, dimethylaminopropyl, CH₂COOMe; CH₂CONH₂, CH₂CONHMe, CH₂CONMe₂, CH₂CN, pyrrolidinylethyl, morpholinylethyl or N-methylpiperazinylethyl.

3. Method as claimed in claim 1, wherein R2 is selected from COOMe, COOEt, or a 5-membered heterocycle containing from 2 to 4 heteroatoms chosen from N and O, or R2 forms, together with R1, a substituted 1,2,3,4-tetrahydro-pyrazino[1,2-a]indole, 3,4-dihydro-2H-pyrazino[1,2-a]indol-1-one, 1,2-dihydro-pyrazino[1,2-a]indol-3-one or 3,4-dihydro-1H-[1,4]oxazino[4,3-a]indole.

4. Method as claimed in claim 1, wherein R3, R4, R5 and R6 independently represent Me, Et, OMe, OEt; Cl or F.

5. Method as claimed in claim 1, for the treatment or prevention of tumours of the digestive system, urinary system, central nervous system, breast, bones and of melanomas.

6. Method as claimed in claim 1, wherein said compound of formula (I) is used as an enhancer of antitumour drugs and/or in the treatment of resistance to antitumour drugs, as an antimetastatic agent and as a radiosensitizer in the radiation therapy.

7. Method as claimed in claim 1, wherein the compound of formula (I) is selected from the group consisting of:
5,6-Dimethoxy-3-(4-methoxy-phenyl)-1H-indole-2-carboxylic acid methylester
3-(3,4-Dimethoxy-phenyl)-5,6-dimethoxy-1H-indole-2-carboxylic acid methylester
5,6-Dimethoxy-3-phenyl-1H-indole-2-carboxylic acid methylester
5,6-Dimethoxy-3-phenyl-1H-indole-2-carboxylic acid ethylester
3-(4-Methoxy phenyl)-1H-indole-2-carboxylic acid methylester
1-Benzyl-5,6-dimethoxy-3-(4-methoxyphenyl)-1H-indole-2-carboxylic acid methylester
5,6-Dimethoxy-1-methoxycarbonylmethyl-3-(4-methoxyphenyl)-1H-indole-2-carboxylic acid methylester
1-Dimethylcarbamoylmethyl-5,6-dimethoxy-3-(4-methoxyphenyl)-1H-indole-2-carboxylic acid methylester
5,6-Dimethoxy-3-(4-methoxyphenyl)-1-propyl-1H-indole-2-carboxylic acid methylester
1-Cyanomethyl-5,6-dimethoxy-3-(4-methoxyphenyl)-1H-indole-2-carboxylic acid methylester
-1-(2-Dimethylaminoethyl)-5,6-dimethoxy-3-(4-methoxyphenyl)-1H-indole-2 carboxylic acid methylester hydrochloride
-1-(2-Hydroxyethyl)-5,6-dimethoxy-3-(4-methoxyphenyl)-1H-indole-2-carboxylic acid methylester
-5,6-Dimethoxy-3-(4-methoxyphenyl)-1H-indole-2-carboxyamide
-N-[5,6-dimethoxy-3-(4-methoxyphenyl)-1H-indol-2-ylmethyl]-acetamide
-5,6-Dimethoxy-3-(4-methoxyphenyl)-1H-indole-2-carbonitrile
-1-(2-Dimethylaminoethyl)-5,6-dimethoxy-3-(4-methoxy-phenyl)-1H-indole-2-carbonitrile hydrochloride
-5,6-Dimethoxy-3-(4-methoxyphenyl)-2-(2H-[1,2,4]triazol-3-yl)-1H-indole
-2-(4,5-Dihydro-1H-imidazol-2-yl)-5,6-dimethoxy-3-(4-methoxy-phenyl)-1H-indole trifluoroacetate
-5,6-Dimethoxy-3-(4-methoxyphenyl)-2-(1H-tetrazol-5-yl)-1H-indole
-5,6-Dimethoxy-3-(4-methoxyphenyl)-2-[1,3,4]oxadiazol-2-yl-1H-indole
-7,8-Dimethoxy-10-(4-methoxyphenyl)-3,4-dihydro-2H-pyrazino[1,2-a]indol-1-one
7,8-Dimethoxy-10-(4-methoxyphenyl)-1,2,3,4-tetrahydro-pyrazino[1,2-a]indole hydrochloride
3-(4-Chlorophenyl)-5,6-dimethoxy-1H-indole-2-carboxylic acid methyl ester
3-(4-(Trifluoromethyl)phenyl)-5,6-dimethoxy-1H-indole-2-carboxylic acid methyl ester
5,6-Dimethoxy-3-p-tolyl-1H-indole-2-carboxylic acid methyl ester
3-(4-Fluorophenyl)-5,6-dimethoxy-1H-indole-2-carboxylic acid methyl ester
3-(2-Chlorophenyl)-5,6-dimethoxy-1H-indole-2-carboxylic acid methyl ester
3-(3-Chlorophenyl)-5,6-dimethoxy-1H-indole-2-carboxylic acid methyl ester
5-Chloro-3-phenyl-1H-indole-2-carboxylic acid ethyl ester
5-Fluoro-3-phenyl-1H-indole-2-carboxylic acid ethyl ester
5-Methoxy-3-phenyl-1H-indole-2-carboxylic acid ethyl ester
5,6-Dimethoxy-1-(2-methoxyethyl)-3-(4-methoxyphenyl)-1H-indole-2-carboxylic acid methyl ester
1-(2-Hydroxyethyl)-5,6-dimethoxy-3-phenyl-1H-indole-2-carbonitrile
1-(3-Hydroxypropyl)-5,6-dimethoxy-3-(4-methoxyphenyl)-1H-indole-2-carboxylic acid methyl ester
1-(2-Hydroxyethyl)-5,6-dimethoxy-3-(4-methoxyphenyl)-1H-indole-2-carbonitrile
2-(5,6-Dimethoxy-3-(4-methoxyphenyl)-2-(5-methyl-1,3,4-oxadiazol-2-yl)-1H-indol-1-yl)ethanol
1-(3-Hydroxypropyl)-5,6-dimethoxy-3-(4-methoxyphenyl)-1H-indole-2-carbonitrile
5,6-Dimethoxy-3-phenyl-1H-indole-2-carboxylic acid amide
5,6-Dimethoxy-3-phenyl-1H-indole-2-carboxylic acid dimethylamide
(5,6-Dimethoxy-3-phenyl-1H-indol-2-yl)-morpholin-4-yl-methanone
5,6-Dimethoxy-3-phenyl-1H-indole-2-carboxylic acid methylamide
5,6-Dimethoxy-3-phenyl-1H-indole-2-carbonitrile
5,6-Dimethoxy-3-phenyl-1-propyl-1H-indole-2-carbonitrile
1-(2-(Dimethylamino)ethyl)-5,6-dimethoxy-3-phenyl-1H-indole-2-carbonitrile hydrochloride
5,6-Dimethoxy-3-(4-methoxyphenyl)-1-(2-(pyrrolidin-1-yl)ethyl)-1H-indole-2-carboxylic acid methyl ester hydrochloride
1-(3-(Dimethylamino)propyl)-5,6-dimethoxy-3-(4-methoxyphenyl)-1H-indole-2-carboxylic acid methyl ester hydrochloride
5,6-Dimethoxy-3-(4-methoxyphenyl)-1-(2-(pyrrolidin-1-yl)ethyl)-1H-indole-2-carbonitrile hydrochloride
1-(3-(Dimethylamino)propyl)-5,6-dimethoxy-3-(4-methoxyphenyl)-1H-indole-2-carbonitrile
5,6-Dimethoxy-3-(4-methoxyphenyl)-1-(2-morpholinoethyl)-1H-indole-2-carbonitrile hydrochloride
2-(5,6-Dimethoxy-3-(4-methoxyphenyl)-2-(5-methyl-1,3,4-oxadiazol-2-yl)-1H-indol-1-yl)-N,N-dimethylethanamine
5,6-Dimethoxy-3-phenyl-2-(4H-1,2,4-triazol-3-yl)-1H-indole
3,4-Dihydro-7,8-dimethoxy-10-phenylpyrazino[1,2-a]indol-1(2H)-one
1-(2-Amino-ethyl)-5,6-dimethoxy-3-(4-methoxy-phenyl)-1H-indole-2-carboxylic acid methyl ester hydrochloride
7,8-Dimethoxy-10-phenyl-1,2,3,4-tetrahydro-pyrazino[1,2-a]indole hydrochloride
5,6-Dimethoxy-3-(4-methoxyphenyl)-1-[2-(4-methylpiperazin-1-yl)-ethyl]-1H-indole-2-carboxylic acid methyl ester dihydrochloride
5,6-Dimethoxy-3-(4-methoxyphenyl)-1-(2-morpholinoethyl)-1H-indole-2-carboxylic acid methyl ester hydrochloride
5,6-Dimethoxy-3-(4-methoxyphenyl)-1-(2-(4-methylpiperazin-1-yl)ethyl)-1H-indole-2-carbonitrile dihydrochloride
7,8-Dimethoxy-10-(4-methoxyphenyl)-2-methyl-1,2,3,4-tetrahydro-pyrazino[1,2-a]indole hydrocloride
7,8-Dimethoxy-10-(4-methoxyphenyl)-1,2-dihydropyrazino[1,2-a]indol-3-one
2-Methanesulfonyl-7,8-dimethoxy-10-(4-methoxyphenyl)-1,2,3,4-tetrahydro-pyrazino[1,2-a]indole
7,8-Dimethoxy-10-(4-methoxyphenyl)-2-(propane-2-sulfonyl)-1,2,3,4-tetrahydropyrazino[1,2-a]indole
7,8-Dimethoxy-10-(4-methoxyphenyl)-2-(toluene-4-sulfonyl)-1,2,3,4-tetrahydropyrazino[1,2-a]indole
1-[7,8-Dimethoxy-10-(4-methoxyphenyl)-3,4-dihydro-1H-pyrazino[1,2-a]indol-2-yl]-ethanone
7,8-Dimethoxy-10-(4-methoxyphenyl)-3,4-dihydro-1H-pyrazino[1,2-a]indole-2-carboxylic acid methylamide
2-Isopropyl-7,8-dimethoxy-10-(4-methoxyphenyl)-1,2,3,4-tetrahydro-pyrazino[1,2-a]indole hydrochloride
1-Carbamoylmethyl-5,6-dimethoxy-3-(4-methoxyphenyl)-1H-indole-2-carboxylic acid methyl ester
2-(4,5-Dihydrooxazol-2-yl)-5,6-dimethoxy-3-phenyl-1H-indole
5,6-Dimethoxy-3-(4-methoxyphenyl)-1-methylcarbamoylmethyl-1H-indole-2-carboxylic acid methyl ester
3,4-Dihydro-7,8-dimethoxy-10-(4-methoxyphenyl)-1H-[1,4]oxazino[4,3-a]indole
5,6-Dimethoxy-3-(4-methoxyphenyl)-2-(5-methyl-1,3,4-oxadiazol-2-yl)-1H-indole
5-Hydroxy-3-phenyl-1H-indole-2-carboxylic acid ethyl estersand
3-Phenyl-1H-indole-2-carboxylic acid ethyl ester.

8. Pharmaceutical composition comprising a compound of formula (I) 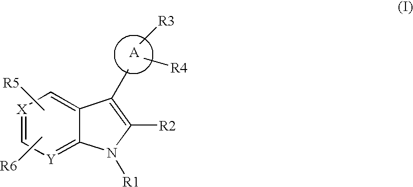
wherein:
R1 is chosen from arylalkyl, hydroxyalkyl, alkoxyalkyl, aminoalkyl, alkylaminoalkyl, dialkylaminoalkyl, alkoxycarbonylalkyl, aminocarbonylalkyl, alkylaminocarbonylalkyl, dialkylaminocarbonylalkyl, cyanoalkyl, or a group R′R″Nalkyl, in which R′ and R″, together with the nitrogen atom to which they are attached, may form a 5, 6 or 7 membered ring, optionally containing a heteroatom chosen from O, S and N, and where said N atom may be substituted by alkyl;
R2 is chosen from heterocyclyl optionally substituted by alkyl or aryl, ester, amide, nitrile; or R1 and R2 together form a 5, 6 or 7 membered ring containing optionally a heteroatom chosen from O, S, N and containing optionally a carbonyl function which can be attached to any carbon atom of said ring, and where said N atom may be substituted by alkyl, aryl, arylalkyl, heteroaryl, alkylsulfonyl, arylsulfonyl, alkylcarbonyl, arylcarbonyl, alkylaminocarbonyl, arylaminocarbonyl;
R3, R4, R5, R6 each independently represent H, alkyl, alkoxy, hydroxy, halogen, trifluoromethyl, trifluoromethyloxy;
X and Y each independently represent carbon;
A is phenyl,
where in all substituents referred above, the term “aryl” means phenyl.

9. Pharmaceutical composition as claimed in claim 8, wherein the further antitumour drug is chosen from anthracyclines, camptothecins, platinum compounds and taxans.

10. Pharmaceutical composition as claimed in claim 8, in the form of an injectable solution, a solution for infusion, a solution for inhalation, a suspension, an emulsion, a syrup, an elixir, drops, a suppository, a possibly coated pill, a hard or soft capsule, a microcapsule, granules or dispersible powders.

11. Pharmaceutical composition as claimed in claim 8, in the form of a dosage unit comprising the compound of formula (I) in a quantity between 1 and 1000 mg.

12. Pharmaceutical composition as claimed in claim 8, in the form of a dosage unit comprising the further antitumour drug in a quantity between 0.1 and 1000 mg.

13. Compound of formula (I) 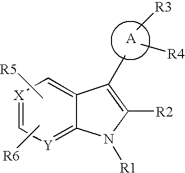
wherein:
R1 is chosen from H, alkyl, arylalkyl, hydroxyalkyl, alkoxyalkyl, dialkylaminoalkyl, alkoxycarbonylalkyl, aminocarbonylalkyl, alkylaminocarbonylalkyl, dialkylaminocarbonylalkyl, or a group R′R″Nalkyl, in which R′ and R″, together with the nitrogen atom to which they are attached, may form a 5, 6 or 7 membered ring, optionally containing a heteroatom chosen from O, S and N, and where said N atom may be substituted by alkyl;
R2 is chosen from heterocyclyl optionally substituted by alkyl or aryl, acid, ester, amide, nitrile; or R1 and R2 together form a 5, 6 or 7 membered ring containing optionally a heteroatom chosen from O, S, N and where said N atom may be substituted by alkyl, aryl, arylalkyl, heteroaryl, alkylsulfonyl, arylsulfonyl, alkylcarbonyl, arylcarbonyl, alkylaminocarbonyl, arylaminocarbonyl;
R3, R4, R5, R6 each independently represent H, alkyl, alkoxy, hydroxy, trifluoromethyl, trifluoromethyloxy;
X and Y each independently represent carbon;
A is phenyl,
where in all substituents referred above, the term “aryl” means phenyl,
with the exception of the compounds in which R1 is H or alkyl and, simultaneously, R2 is chosen from acid, ester, amide or hydroxyalkyl, and with exception of the following specific compounds a-f:
of formula (II): 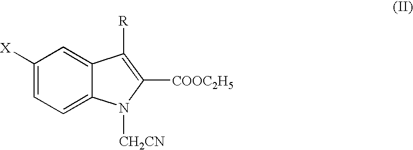
wherein:
R═C₆H₅,X═Cl a) 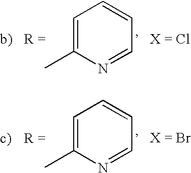
wherein:
R═C₆H₅,X═Cl d)

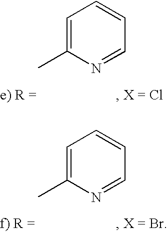

14. Compound as claimed in claim 13, chosen from the group consisting of:
1-Benzyl-5,6-dimethoxy-3-(4-methoxy-phenyl)-1H-indole-2-carboxylic acid methylester
5,6-Dimethoxy-1-methoxycarbonylmethyl-3-(4-methoxy-phenyl)-1H-indole-2-carboxylic acid methylester
1-Dimethylcarbamoylmethyl-5,6-dimethoxy-3-(4-methoxy-phenyl)-1H-indole-2-carboxylic acid methylester
[1-Cyanomethyl-5,6-dimethoxy-3-(4-methoxy-phenyl)-1H-indole-2-carboxylic acid methylester]
1-(2-Dimethylamino-ethyl)-5,6-dimethoxy-3-(4-methoxy-phenyl)-1H-indole-2-carboxylic acid methylester hydrochloride
1-(2-Hydroxy-ethyl)-5,6-dimethoxy-3-(4-methoxy-phenyl)-1H-indole-2-carboxylic acid methylester
N-[5,6-dimethoxy-3-(4-methoxy-phenyl)-1H-indol-2-ylmethyl]-acetamide
5,6-Dimethoxy-3-(4-methoxy-phenyl)-1H-indole-2-carbonitrile
1-(2-Dimethylamine-ethyl)-5,6-dimethoxy-3-(4-methoxy-phenyl)-1H-indole-2-carbonitrile hydrochloride
5,6-Dimethoxy-3-(4-methoxy-phenyl)-2-(2H-[1,2,4]triazol-3-yl)-1H-indole
2-(4,5-Dihydro-1H-imidazol-2-yl)-5,6-dimethoxy-3-(4-methoxy-phenyl)-1H-indole trifluoroacetate
5,6-Dimethoxy-3-(4-methoxy-phenyl)2-(1H-tetrazol-5-yl)-1H-indole
5,6-Dimethoxy-3-(4-methoxy-phenyl)-2-[1,3,4]oxadiazol-2-yl-1H-indole
7,8-Dimethoxy-10-(4-methoxy-phenyl)-3,4-dihydro-2H-pyrazino[1,2-a]indol-1-one
7,8-Dimethoxy-10-(4-methoxy-phenyl)-1,2,3,4-tetrahydro-pyrazino[1,2-a]indole hydrochloride
5,6-Dimethoxy-1-(2-methoxy-ethyl)-3-(4-methoxy-phenyl)-1H-indole-2-carboxylic acid methyl ester
1-(2-Hydroxyethyl)-5,6-dimethoxy-3-phenyl-1H-indole-2-carbonitrile
1-(3-Hydroxypropyl)-5,6-dimethoxy-3-(4-methoxyphenyl)-1H-indole-2-carboxylic acid methyl ester
1-(2-Hydroxyethyl)-5,6-dimethoxy-3-(4-methoxyphenyl)-1H-indole-2-carbonitrile
2-(5,6-Dimethoxy-3-(4-methoxyphenyl)-2-(5-methyl-1,3,4-oxadiazol-2-yl)-1H-indol-1-yl)ethanol
1-(3-Hydroxypropyl)-5,6-dimethoxy-3-(4-methoxyphenyl)-1H-indole-2-carbonitrile
(5,6-Dimethoxy-3-phenyl-1H-indol-2-yl)-morpholin-4-yl-methanone
5,6-Dimethoxy-3-phenyl-1H-indole-2-carbonitrile
5,6-Dimethoxy-3-phenyl-1-propyl-1H-indole-2-carbonitrile
1-(2-(Dimethylamino)ethyl)-5,6-dimethoxy-3-phenyl-1H-indole-2-carbonitrile hydrochloride
5,6-Dimethoxy-3-(4-methoxyphenyl)-1-(2-(pyrrolidin-1-yl)ethyl)-1H-indole-2-carboxylic acid methyl ester hydrochloride
1-(3-(Dimethylamino)propyl)-5,6-dimethoxy-3-(4-methoxyphenyl)-1H-indole-2-carboxylic acid methyl ester hydrochloride
5,6-Dimethoxy-3-(4-methoxyphenyl)-1-(2-(pyrrolidin-1-yl)ethyl)-1H-indole-2-carbonitrile hydrochloride
1-(3-(Dimethylamino)propyl)-5,6-dimethoxy-3-(4-methoxyphenyl)-1H-indole-2-carbonitrile
5,6-Dimethoxy-3-(4-methoxyphenyl)-1-(2-morpholinoethyl)-1H-indole-2-carbonitrile hydrochloride
2-(5,6-Dimethoxy-3-(4-methoxyphenyl)-2-(5-methyl-1,3,4-oxadiazol-2-yl)-1H-indol-1-yl)-N,N-dimethylethanamine
5,6-Dimethoxy-3-phenyl-2-(4H-1,2,4-triazol-3-yl)-1H-indole
3,4-Dihydro-7,8-dimethoxy-10-phenylpyrazino[1,2-a]indol-1(2H)-one
[1-(2-Amino-ethyl)-5,6-dimethoxy-3-(4-methoxy-phenyl)-1H-indole-2-carboxylic acid methyl ester hydrochloride]
7,8-Dimethoxy-10-phenyl-1,2,3,4-tetrahydro-pyrazino[1,2-a]indole hydrochloride
5,6-Dimethoxy-3-(4-methoxy-phenyl)-1-[2-(4-methyl-piperazin-1-yl)-ethyl]-1H-indole-2-carboxylic acid methyl ester dihydrochloride
5,6-Dimethoxy-3-(4-methoxyphenyl)-1-(2-morpholinoethyl)-1H-indole-2-carboxylic acid methyl ester hydrochloride
5,6-Dimethoxy-3-(4-methoxyphenyl)-1-(2-(4-methylpiperazin-1-yl)ethyl)-1H-indole-2-carbonitrile dihydrochloride
7,8-Dimethoxy-10-(4-methoxy-phenyl)-2-methyl-1,2,3,4-tetrahydro-pyrazino[1,2-a]indole hydrocloride
7,8-Dimethoxy-10-(4-methoxy-phenyl)-1,2-dihydro-pyrazino[1,2-a]indol-3-one
2-Methanesulfonyl-7,8-dimethoxy-10-(4-methoxy-phenyl)-1,2,3,4-tetrahydro-pyrazino[1,2-a]indole
7,8-Dimethoxy-10-(4-methoxy-phenyl)-2-(propane-2-sulfonyl)-1,2,3,4-tetrahydro-pyrazino[1,2-a]indole
7,8-Dimethoxy-10-(4-methoxy-phenyl)-2-(toluene-4-sulfonyl)-1,2,3,4-tetrahydro-pyrazino[1,2-a]indole
1-[7,8-Dimethoxy-10-(4-methoxy-phenyl)-3,4-dihydro-1H-pyrazino[1,2-a]indol-2-yl]-ethanone
7,8-Dimethoxy-10-(4-methoxy-phenyl)-3,4-dihydro-1H-pyrazino[1,2-a]indole-2-carboxylic acid methylamide
2-Isopropyl-7,8-dimethoxy-10-(4-methoxy-phenyl)-1,2,3,4-tetrahydro-pyrazino[1,2-a]indole hydrochloride
1-Carbamoylmethyl-5,6-dimethoxy-3-(4-methoxy-phenyl)-1H-indole-2-carboxylic acid methyl ester
2-(4,5-Dihydrooxazol-2-yl)-5,6-dimethoxy-3-phenyl-1H-indole
5,6-Dimethoxy-3-(4-methoxy-phenyl)-1-methylcarbamoylmethyl-1H-indole-2-carboxylic acid methyl ester
3,4-Dihydro-7,8-dimethoxy-10-(4-methoxyphenyl)-1H-[1,4]oxazino[4,3-a]indole, and
5,6-Dimethoxy-3-(4-methoxyphenyl)-2-(5-methyl-1,3,4-oxadiazol-2-yl)-1H-indole.

15. (canceled)

16. Pharmaceutical composition containing a compound of formula (I) as defined in claim 13, in association with suitable pharmaceutical excipients, and optionally further antitumour drugs.

17. Method for treating or preventing tumours comprising administering to a patient in need thereof a compound of formula (I) as defined in claim 13.
